(19) United States
(12) Patent Application Publication
(10) Pub. No.: US 2026/0035730 A1
(43) Pub. Date: Feb. 5, 2026

(54) CELL LYSIS AND NUCLEIC ACID RECOVERY

(71) Applicant: BioFire Diagnostics, LLC, Salt Lake City, UT (US)

(72) Inventors: Erik Wong Huynh, West Jordan, UT (US); Andrew Carter Hatch, West Jordan, UT (US); Stephanie Anne Thatcher, Salt Lake City, UT (US)

(21) Appl. No.: 18/997,459

(22) PCT Filed: Jul. 18, 2023
(86) PCT No.: PCT/US2023/070446

§ 371 Date: Jan. 21, 2025

Related U.S. Application Data
(60) Provisional application No. 63/369,107, filed on Jul. 22, 2022.
(60) Provisional application No. 63/450,492, filed on Mar. 7, 2023.

(51) Int. Cl. C12Q 1/6806; C12Q 1/6844

(52) U.S. Cl. CPC C12Q 1/6806; C12Q 1/6844

(57) ABSTRACT

Methods and systems for rapid preparation of a nucleic acid sample.

### CROSS REFERENCE TO RELATED APPLICATIONS

This application claims the benefit of and priority to U.S. Prov. Pat. App. No. 63/369,107 filed 22 Jul. 2022 and U.S. Prov. Pat. App. No. 63/450,492 filed 7 Mar. 2023, the entireties of which are incorporated herein by reference.

### BACKGROUND OF THE INVENTION

In the United States, Canada, and Western Europe infectious disease accounts for approximately 7% of human mortality, while in developing regions infectious disease accounts for over 40% of human mortality. Infectious diseases lead to a variety of clinical manifestations. Among common overt manifestations are fever, pneumonia, meningitis, diarrhea, and diarrhea containing blood. While the physical manifestations suggest some pathogens and eliminate others as the etiological agent, a variety of potential causative agents remain, and clear diagnosis often requires a variety of assays to be performed. Traditional microbiology techniques for diagnosing pathogens can take days or weeks, often delaying a proper course of treatment.

In recent years, the polymerase chain reaction (PCR) has become a method of choice for rapid diagnosis of infectious agents. PCR can be a rapid, sensitive, and specific tool to diagnose infectious disease. A challenge to using PCR as a primary means of diagnosis is the variety of possible causative organisms and the low levels of organism present in some pathological specimens. It is often impractical to run large panels of PCR assays, one for each possible causative organism, most of which are expected to be negative. The problem is exacerbated when pathogen nucleic acid is at low concentration and requires a large volume of sample to gather adequate reaction templates. In some cases, there is inadequate sample to assay for all possible etiological agents. A solution is to run “multiplex PCR” wherein the sample is concurrently assayed for multiple targets in a single reaction. While multiplex PCR has proven to be valuable in some systems, shortcomings exist concerning robustness of high-level multiplex reactions and difficulties for clear analysis of multiple products. To solve these problems, the assay may be subsequently divided into multiple secondary PCRs. Nesting secondary reactions within the primary product often increases robustness. However, this further handling can be expensive and may lead to contamination or other problems.

The FilmArray® (BioFire Diagnostics, LLC, Salt Lake City, UT) is a user friendly, highly multiplexed PCR system developed for the diagnostic market. The single sample instrument accepts a diagnostic “pouch” that integrates sample preparation and nested multiplex PCR. Integrated sample preparation provides ease-of-use, while the highly multiplexed PCR provides both the sensitivity of PCR and the ability to test for many organisms simultaneously (e.g., up to 30 or more different organism and molecular markers). This system is well suited to pathogen identification where a number of different pathogens all manifest similar clinical symptoms. Current available diagnostic panels include a respiratory panel for upper respiratory infections, a blood culture panel for blood stream infections, a gastrointestinal panel for GI infections, a meningitis/encephalitis panel for central nervous system infections, a pneumonia panel for lower respiratory infections, and a bone and joint panel for bone and joint infections. Other panels are in development.

When PCR was first popularized in the late 1980s, the process was slow. A typical protocol was one minute for denaturation at 94° C., two minutes for annealing at 55° C., and three minutes for extension at 72° C. When the time for transition between temperatures was included, 8-minute cycles were typical, resulting in completion of 30 cycles in four hours. Over the years, systems have become faster, thirty-minute protocols are common, and faster PCR devices are available. Even with faster PCR protocols, many diagnostic PCR reactions begin with target cell lysis and extraction/purification of target nucleic acids from the target cells using older techniques.

As PCR gets faster, the time spent in sample preparation becomes an increasingly larger portion of the processing time. One possible solution would be to essentially skip sample preparation, relying on chemical and/or heated lysis to disrupt cells and to perform nucleic acid amplification in raw lysate. This does save time, but sufficiency of lysis is not always reliable and there are many potential inhibitors of nucleic acid amplification in unpurified lysate. Another possible solution involves mechanical lysis followed by recovery of nucleic acids from the lysate and purification of the nucleic acids. While this is desirable from the perspective of the cleanliness of the starting template for the nucleic acid amplification, it has traditionally been quite slow. It would be desirable to perform sample preparation using faster methods while maximizing the cleanliness of the starting template for the nucleic acid amplification. There exists a need in the art for retaining the cleanliness and robustness of traditional sample preparation (i.e., mechanical lysis, recovery of nucleic acids, and purification of the nucleic acids prior to starting nucleic acid amplification) while also shortening the time needed for sample preparation.

### BRIEF SUMMARY

Described herein are methods and systems for cell lysis and nucleic acid recovery. The methods include combining lysis particles (e.g., zirconium silicate beads) and a first quantity of nucleic acid binding magnetic particles (e.g., silica-coated magnetic beads) with a sample suspected of containing one or more target nucleic acids and a lysis buffer in a container (e.g., a tube). The container then suitably may be placed in a bead beater for a sufficient time to produce a lysate. After bead beating to produce a lysate, a second quantity of nucleic acid binding magnetic particles suitably may be mixed into the lysate. After mixing the second quantity of nucleic acid binding magnetic particles into the lysate, the first and second quantities of nucleic acid binding magnetic particles may be recovered from the lysate—illustratively with a magnet.

After recovery, the magnetic particles may be transferred to another container, released, and washed one or more times to remove lysate residue. The magnetic particles may then be recaptured (illustratively with the magnet), the wash buffer may be removed, the magnetic particles may be released again and mixed with an elution buffer to elute the captured nucleic acids from the magnetic particles. The magnetic particles may be captured again, and the elution buffer may be recovered and transferred to a clean tube. Alternatively, the magnet may be used to remove the magnetic particle from the tube and the magnetic particles may be disposed of. The eluted materials may be used for a variety of downstream assays, such as, but not limited to, assaying the eluted materials for presence of the one or more target nucleic acids suspected to be in the sample with one or more of nucleic acid amplification and detection, sequencing, next generation sequencing, and the like. That is, if the nucleic acids in the sample are from cells (e.g., pathogen cells), the assay may be used to identify the cells that the nucleic acids are derived from.

In one example, a method for cell lysis and nucleic acid recovery is disclosed. The method includes steps of providing a sample container, disposing a quantity of lysis particles in the sample container, adding a sample suspected of containing one or more target nucleic acids and a lysis buffer to the container, adding a first quantity of nucleic acid binding magnetic particles to the container, agitating the container with the lysis particles, the sample, the lysis buffer, and the first quantity of nucleic acid binding magnetic particles for a first period of time to generate a lysate, mixing a second quantity of nucleic acid binding magnetic particles into the lysate in the container, and recovering the first and second quantities of nucleic acid binding magnetic particles from the lysate.

In another example, a method for cell lysis and nucleic acid recovery is disclosed. The method includes steps of providing a sample container comprising a first chamber and a second chamber, combining in the first chamber a quantity of lysis particles, a first quantity nucleic acid binding magnetic particles, a sample suspected of containing one or more target nucleic acids, and a lysis buffer, agitating the first chamber of the sample container with the lysis particles, the sample, the lysis buffer, and the first quantity of nucleic acid binding magnetic particles for a period of time sufficient to generate a lysate, mixing a second quantity of nucleic acid binding magnetic particles into the lysate, and capturing the first and second quantities of nucleic acid binding magnetic particles from the lysate in the second chamber using a magnet.

In yet another example, a method for cell lysis and nucleic acid recovery is disclosed. The method includes steps of providing a sample container comprising a plurality of fluidly connected reaction chambers including a sample lysis chamber, a nucleic acid recovery chamber, and at least a first nucleic acid amplification chamber, combining in the sample lysis chamber a quantity of lysis particles, a first quantity nucleic acid binding magnetic particles, a sample suspected of containing one or more target nucleic acids, and a lysis buffer, agitating the sample lysis chamber of the sample container with the lysis particles, the sample, the lysis buffer, and the first quantity of nucleic acid binding magnetic particles for a period of time sufficient to generate a lysate, mixing a second quantity of nucleic acid binding magnetic particles into the lysate, and, using a magnet, capturing the first and second quantities of nucleic acid binding magnetic particles from the lysate in the nucleic acid recovery chamber. The method further includes releasing the magnet and washing the first and second quantities of nucleic acid binding magnetic particles with a wash buffer in the nucleic acid recovery chamber, recapturing the first and second quantities of nucleic acid binding magnetic particles with the magnet and removing the wash buffer, releasing the magnet and eluting the nucleic acids from the first and second quantities of nucleic acid binding magnetic particles with an elution buffer in the nucleic acid recovery chamber, and recapturing the first and second quantities of nucleic acid binding magnetic particles with the magnet and transferring the elution buffer to the first nucleic acid amplification chamber.

In one aspect of the methods disclosed herein, adding the first quantity of nucleic acid binding magnetic particles and then the second quantity of nucleic acid binding magnetic particles is selected to boost recovery of RNA and DNA from the sample. Such a recovery boost may, for example, be expressed in terms of Cp improvements (as shown by an earlier Cp) or improvements to test sensitivity.

Described herein are:
A1. A method for cell lysis and nucleic acid recovery, comprising:
providing a sample container, a quantity of lysis particles, a sample suspected of containing one or more target nucleic acids, a lysis buffer, and a first quantity of nucleic acid binding magnetic particles,
disposing in the container the sample, the quantity of lysis particles, the lysis buffer, and the first quantity of nucleic acid binding magnetic particles,
agitating the container with the lysis particles, the sample, the lysis buffer, and the first quantity of nucleic acid binding magnetic particles for a first period of time to generate a lysate,
dispersing a second quantity of nucleic acid binding magnetic particles into the lysate in the container, and
recovering the first and second quantities of nucleic acid binding magnetic particles from the lysate,
wherein adding the first quantity of nucleic acid binding magnetic particles and then the second quantity of nucleic acid binding magnetic particles is selected to boost recovery of nucleic acids from the sample.
A2. The method of clause A1, wherein the lysis buffer comprises a buffering agent, a chaotropic salt, and a non-ionic surfactant.
A2.1 The method of clause A1 or A2, wherein the lysis buffer is an aqueous buffer comprising the buffering agent, 50-60% of the chaotropic agent, and 10-20% of the non-ionic surfactant.
A2.2 The method of clause A1, A2, or A2.1, wherein the chaotropic agent is a guanidinium salt and the non-ionic surfactant is one of Triton X-100, polidocanol (Thesit), Triton X-114, NP-40, Arlasolve 200, Brij 010, octyl β-D-glucopyranoside, a saponin, nonaethylene glycol monododecyl ether, and combinations thereof.
A3. The method of any one of clauses A1-A2.2, wherein the recovering further comprises one of releasing the first and second quantities of nucleic acid binding magnetic particles recovered from the lysate into another sample container, or removing the lysate and lysis particles from the container and releasing the first and second quantities of nucleic acid binding magnetic particles recovered from the lysate back into the container.
A4. The method of any one of clauses A1-A3, further comprising washing the first and second quantities of nucleic acid binding magnetic particles with a wash buffer, recapturing the first and second quantities of nucleic acid binding magnetic particles with the magnet, and removing the wash buffer.
A5. The method of any one of clauses A1-A4, wherein the washing does not include one or more of heating the wash buffer and magnetic particles prior to or during the washing, aggressively mixing the magnetic particles and the wash buffer, or incubating the magnetic particles and the wash buffer for a period of time greater than 10 seconds.
A6. The method of any one of clauses A1-A5, further comprising releasing the first and second quantities of nucleic acid binding magnetic particles from the magnet, adding an elution buffer to the first and second quantities of nucleic acid binding magnetic particles, releasing the magnet, and mixing the magnetic particles with the elution buffer, recapturing the first and second quantities of nucleic acid binding magnetic particles with the magnet, and transferring the elution buffer to another sample container.
A7. The method of any one of clauses A1-A6, further comprising adding an elution buffer to the first and second quantities of nucleic acid binding magnetic particles and mixing the magnetic particles with the elution buffer, recapturing the first and second quantities of nucleic acid binding magnetic particles with the magnet, and transferring the first and second quantities of nucleic acid binding magnetic particles to another sample container.
A8. The method of any one of clauses A1-A7, wherein the elution buffer is configured to elute nucleic acids captured by the nucleic acid binding magnetic particles into the elution buffer.
A9. The method of any one of clauses A1-A8, wherein an amount of the second quantity of nucleic acid binding magnetic particles is substantially equal to an amount of the first quantity of nucleic acid binding magnetic particles.
A10. The method of any one of clauses A1-A9, further comprising assaying the elution buffer for presence of the one or more target nucleic acids suspected to be in the sample.
A11. The method of any one of clauses A1-A10, wherein the assaying comprises a nucleic acid amplification step and a step of detection amplified nucleic acids.
A12. The method of any one of clauses A1-A11, wherein the agitating step comprises heating the sample and the dispersing step comprises cooling the sample.
B1. A method for cell lysis and nucleic acid recovery, comprising:
providing a sample container,
combining in the sample container a quantity of lysis particles, a first quantity nucleic acid binding magnetic particles, a sample suspected of containing one or more target nucleic acids, and a lysis buffer,
agitating the lysis particles, the sample, the lysis buffer, and the first quantity of nucleic acid binding magnetic particles in the sample container for a period of time sufficient to generate a lysate,
dispersing a second quantity of nucleic acid binding magnetic particles into the lysate in the sample container,
incubating the first and second quantities of nucleic acid binding magnetic particles in the lysate for a period of time, and
capturing the first and second quantities of nucleic acid binding magnetic particles from the lysate using a magnet,
wherein adding the first quantity of nucleic acid binding magnetic particles and then the second quantity of nucleic acid binding magnetic particles is selected to boost recovery of nucleic acids from the sample.
B2. The method of clause B1, wherein the period of time of incubating the first and second quantities of nucleic acid binding magnetic particles in the lysate is in a range of 1 second to 1 minute.
B3. The method of clause B1 or B2, wherein the period of time is in a range of 20-30 seconds.
B4. The method of any one of clauses B1-B3, wherein capturing the first and second quantities of magnetic particles from the lysate further comprises transferring the magnetic particles to a second sample container and releasing the magnetic particles from the magnet into the second sample container, or removing the lysate and the lysis particles from the first sample container and releasing the magnetic particles from the magnet back into the first sample container.
B5. The method of any one of clauses B1-B4, wherein capturing the first and second quantities of magnetic particles from the lysate preferably does not include capturing the lysis particles.
B6. The method of any one of clauses B1-B5, further comprising releasing the first and second quantities of nucleic acid binding magnetic particles from the magnet, adding a wash buffer to the magnetic particles, washing the magnetic particles with the wash buffer, recapturing the magnetic particles with the magnet, removing the wash buffer, adding an elution buffer to the magnetic particles, releasing the magnetic particles from the magnet, mixing the magnetic particles with the elution buffer, recapturing the magnetic particles with the magnet, and transferring either the magnetic particles or the elution buffer to another sample container.
B7. The method of any one of clauses B1-B6, wherein the washing does not include one or more of heating the wash buffer and magnetic particles prior to or during the washing, aggressively mixing the magnetic particles and the wash buffer, or incubating the magnetic particles and the wash buffer for a period of time greater than 10 seconds.
B8. The method of any one of clauses B1-B7, wherein the steps of the method are completed in <4 minutes.
B9. The method of any one of clauses B1-B8, wherein the steps of the method are completed in <3 minutes.
B10. The method of any one of clauses B1-B9, wherein the steps of the method are completed in <2 minutes.
B11. The method of any one of clauses B1-B10, wherein the steps of the method are completed in 1-3 minutes.
C1. A method for cell lysis and nucleic acid recovery, comprising:
providing a sample container comprising a plurality of fluidly connected reaction chambers including a sample lysis chamber, a nucleic acid recovery chamber, and at least a first nucleic acid amplification chamber,
combining in the sample lysis chamber a quantity of lysis particles, a first quantity nucleic acid binding magnetic particles, a sample suspected of containing one or more target nucleic acids, and a lysis buffer,
agitating the lysis particles, the sample, the lysis buffer, and the first quantity of nucleic acid binding magnetic particles in the lysis chamber for a period of time sufficient to generate a lysate,
mixing a second quantity of nucleic acid binding magnetic particles into the lysate,
transferring at least a portion of the lysate having the first and second quantities of nucleic acid binding magnetic particles suspended therein to the nucleic acid recovery chamber,
using a magnet, capturing the first and second quantities of nucleic acid binding magnetic particles from the lysate in the nucleic acid recovery chamber,
removing the lysate but not the first and second quantities of nucleic acid binding magnetic particles from the nucleic acid recovery chamber,
releasing the first and second quantities of nucleic acid binding magnetic particles from the magnet and washing the first and second quantities of nucleic acid binding magnetic particles with a wash buffer in the nucleic acid recovery chamber,
recapturing the first and second quantities of nucleic acid binding magnetic particles with the magnet and removing the wash buffer,
releasing the magnet and mixing the first and second quantities of nucleic acid binding magnetic particles with an elution buffer in the nucleic acid recovery chamber to elute the nucleic acids from the magnetic particles,
recapturing the first and second quantities of nucleic acid binding magnetic particles with the magnet and transferring the elution buffer to the first nucleic acid amplification chamber,
wherein adding the first quantity of nucleic acid binding magnetic particles and then the second quantity of nucleic acid binding magnetic particles is selected to boost recovery of nucleic acids from the sample.
C2. The method of clause C1, wherein capturing the nucleic acid binding magnetic particles comprises deploying a magnet adjacent to the nucleic acid recovery chamber to contain the magnetic particles in that chamber.
C3. The method of clause C1 or C2, further comprising combining the elution buffer with reagents for a nucleic acid amplification reaction to form an amplification mix in the first nucleic acid amplification chamber and subjecting the amplification mix to amplification conditions to assay for presence of the one or more target nucleic acids suspected to be in the sample.
C4. The method of any one of clauses C1-C3, wherein the assay is selected from the group consisting of nucleic acid amplification, sequencing, and next generation sequencing.
C5. The method of any one of clauses C1-C4, wherein the second quantity of nucleic acid binding magnetic particles is substantially equal to the first quantity of nucleic acid binding magnetic particles.
C6. The method of any one of clauses C1-C5, wherein the second quantity of nucleic acid binding magnetic particles is substantially greater than the first quantity of nucleic acid binding magnetic particles.
C7. The method of any one of clauses C1-C6, wherein the second quantity of nucleic acid binding magnetic particles is substantially less than the first quantity of nucleic acid binding magnetic particles.
C8. The method of any one of clauses C1-C7, wherein the agitating step comprises heating the sample and the mixing step comprises cooling the sample.
C9. The method of any one of clauses C1-C8, wherein the washing does not include one or more of heating the wash buffer and magnetic particles prior to or during the washing, aggressively mixing the magnetic particles and the wash buffer, or incubating the magnetic particles and the wash buffer for a period of time greater than 10 seconds.
C10. The method of any one of clauses C1-C9, wherein the steps of the method are completed in <4 minutes.
C11. The method of any one of clauses C1-C10, wherein the steps of the method are completed in <3 minutes.
C12. The method of any one of clauses C1-C11, wherein the steps of the method are completed in <2 minutes.
C13. The method of any one of clauses C1-C12, wherein the steps of the method are completed in 1-3 minutes.

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Additional features and advantages will be set forth in the description that follows, and in part will be obvious from the description, or may be learned by the practice of the invention. The features and advantages may be realized and obtained by means of the instruments and combinations particularly pointed out in the appended claims. These and other features will become more fully apparent from the following description and appended claims, or may be learned by the practice of the invention as set forth hereinafter.

### BRIEF DESCRIPTION OF THE FIGURES

FIG. 1 shows a flexible pouch useful for self-contained PCR.

FIG. 2 is an exploded perspective view of an instrument for use with the pouch of FIG. 1, including the pouch of FIG. 1.

FIG. 3 shows the pouch of FIG. 1 along with the bladder components of FIG. 2.

FIG. 4 shows a motor used in one illustrative embodiment of the instrument of FIG. 2.

FIGS. 5A-5E illustrate steps of a method for cell lysis and nucleic acid recovery.

FIG. 6A is a bar graph illustrating detection of DNA organisms at various concentrations with (BB w/MB) or without (BB w/o MB) silica-coated magnetic particles present during bead beating lysis.

FIG. 6B is a bar graph illustrating detection of RNA organisms at various concentrations with (BB w/MB) or without (BB w/o MB) silica-coated magnetic particles present during bead beating lysis.

FIG. 7A compares crossing points (Cps) for control assays where magnetic particles are added after lysis (w/o Split Magbeads) to assays where a first quantity of magnetic particles were added to the lysis and a second quantity of magnetic particles were added after lysis (w/Split Magbeads).

FIG. 7B illustrates data similar to FIG. 7A for DNA assays.

FIG. 7C illustrates data similar to FIG. 7A for RNA assays.

FIG. 8 illustrates a comparison of Cps for amplification of nucleic acids recovered from magnetic particles included in the lysis with no washes, one wash, or two washes.

FIG. 9 shows fragment sizes of detected human genomic DNA with or without bead beating for 120 seconds.

FIG. 10A illustrates the average Cp response of elution temperature for DNA and RNA assays.

FIGS. 10B and 10C illustrate the Cp response of elution temperature for DNA and RNA assays, respectively, with lines of best fit showing the trend in the data.

FIG. 11 is a bar chart illustrating specific examples of sample preparation times.

### DETAILED DESCRIPTION

Example embodiments are described below with reference to the accompanying drawings. Many different forms and embodiments are possible without deviating from the spirit and teachings of this disclosure and so the disclosure should not be construed as limited to the example embodiments set forth herein. Rather, these example embodiments are provided so that this disclosure will be thorough and complete, and will convey the scope of the disclosure to those skilled in the art. In the drawings, the sizes and relative sizes of layers and regions may be exaggerated for clarity. Like reference numbers refer to like elements throughout the description.

Unless defined otherwise, all terms (including technical and scientific terms) used herein have the same meaning as commonly understood by one of ordinary skill in the art to which the present disclosure pertains. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description of the invention herein is for the purpose of describing particular embodiments only and is not intended to be limiting of the invention. While a number of methods and materials similar or equivalent to those described herein can be used in the practice of the present disclosure, only certain exemplary materials and methods are described herein.

All publications, patent applications, patents or other references mentioned herein are incorporated by reference for in their entirety. In case of a conflict in terminology, the present specification is controlling.

Various aspects of the present disclosure, including devices, systems, methods, etc., may be illustrated with reference to one or more exemplary implementations. As used herein, the terms “exemplary” and “illustrative” mean “serving as an example, instance, or illustration,” and should not necessarily be construed as preferred or advantageous over other implementations disclosed herein. In addition, reference to an “implementation” or “embodiment” of the present disclosure or invention includes a specific reference to one or more embodiments thereof, and vice versa, and is intended to provide illustrative examples without limiting the scope of the invention, which is indicated by the appended claims rather than by the following description.

It will be noted that, as used in this specification and the appended claims, the singular forms “a,” “an,” and “the” include plural referents unless the content clearly dictates otherwise. Thus, for example, reference to “a tile” includes one, two, or more tiles. Similarly, reference to a plurality of referents should be interpreted as comprising a single referent and/or a plurality of referents unless the content and/or context clearly dictate otherwise. Thus, reference to “tiles” does not necessarily require a plurality of such tiles. Instead, it will be appreciated that independent of conjugation; one or more tiles are contemplated herein.

As used throughout this application the words “can” and “may” are used in a permissive sense (i.e., meaning having the potential to), rather than the mandatory sense (i.e., meaning must). Additionally, the terms “including,” “having,” “involving,” “containing,” “characterized by,” variants thereof (e.g., “includes,” “has,” “involves,” “contains,” etc.), and similar terms as used herein, including the claims, shall be inclusive and/or open-ended, shall have the same meaning as the word “comprising” and variants thereof (e.g., “comprise” and “comprises”), and do not exclude additional, un-recited elements or method steps, illustratively.

As used herein, directional and/or arbitrary terms, such as “top,” “bottom,” “left,” “right,” “up,” “down,” “upper,” “lower,” “inner,” “outer,” “internal,” “external,” “interior,” “exterior,” “proximal,” “distal,” “forward,” “reverse,” and the like can be used solely to indicate relative directions and/or orientations and may not be otherwise intended to limit the scope of the disclosure, including the specification, invention, and/or claims.

It will be understood that when an element is referred to as being “coupled,” “connected,” or “responsive” to, or “on,” another element, it can be directly coupled, connected, or responsive to, or on, the other element, or intervening elements may also be present. In contrast, when an element is referred to as being “directly coupled,” “directly connected,” or “directly responsive” to, or “directly on,” another element, there are no intervening elements present.

Example embodiments of the present inventive concepts are described herein with reference to cross-sectional illustrations that are schematic illustrations of idealized embodiments (and intermediate structures) of example embodiments. As such, variations from the shapes of the illustrations as a result, for example, of manufacturing techniques and/or tolerances, are to be expected. Thus, example embodiments of the present inventive concepts should not be construed as limited to the particular shapes of regions illustrated herein but are to include deviations in shapes that result, for example, from manufacturing. Accordingly, the regions illustrated in the figures are schematic in nature and their shapes are not intended to illustrate the actual shape of a region of a device and are not intended to limit the scope of example embodiments.

It will be understood that although the terms “first,” “second,” etc. may be used herein to describe various elements, these elements should not be limited by these terms. These terms are only used to distinguish one element from another. Thus, a “first” element could be termed a “second” element without departing from the teachings of the present embodiments.

It is also understood that various implementations described herein can be utilized in combination with any other implementation described or disclosed, without departing from the scope of the present disclosure. Therefore, products, members, elements, devices, apparatuses, systems, methods, processes, compositions, and/or kits according to certain implementations of the present disclosure can include, incorporate, or otherwise comprise properties, features, components, members, elements, steps, and/or the like described in other implementations (including systems, methods, apparatus, and/or the like) disclosed herein without departing from the scope of the present disclosure. Thus, reference to a specific feature in relation to one implementation should not be construed as being limited to applications only within that implementation.

The headings used herein are for organizational purposes only and are not meant to be used to limit the scope of the description or the claims. To facilitate understanding, like reference numerals have been used, where possible, to designate like elements common to the figures. Furthermore, where possible, like numbering of elements have been used in various figures. Furthermore, alternative configurations of a particular element may each include separate letters appended to the element number.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 5%. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

The word “or” as used herein means any one member of a particular list and also includes any combination of members of that list.

In one aspect, as described in further detail herein, microorganisms from a sample or growth medium can be separated and interrogated to characterize and/or identify the microorganism present in the sample. As used herein, the term “separate” is intended to encompass any sample of microorganisms that has been removed, concentrated or otherwise set apart from its original state, or from a growth or culture medium. For example, in accordance with this invention, microorganisms may be separated away (e.g., as a separated sample) from non-microorganism or non-microorganism components that may otherwise interfere with characterization and/or identification. The term may include microorganisms that have been separated from a mixture by centrifugation, filtration, or any other separation technique known in the art. As such, a separated microorganism sample may include collection of microorganisms and/or components thereof that are more concentrated than, or otherwise set apart from, the original sample, and can range from a closely packed dense clump of microorganisms to a diffuse layer of microorganisms. Non-microorganism components that are separated away from the microorganisms may include non-microorganism cells (e.g., blood cells and/or other tissue cells) and/or any components thereof. In one aspect, the microorganisms are separated from a lysate mixture that includes lysed non-microorganism cells and substantially intact microorganism cells.

In some embodiments, separation of a sample of microorganisms from its original state, or from a growth or culture medium is incomplete. In other words, removing, concentrating, or otherwise setting the microorganisms apart from its original state does not completely separate the sample of microorganisms from other constituents of the sample or from the growth or culture medium. In some cases, a de minimis amount of debris from the sample or from the growth or culture medium is present. For example, the amount of debris or growth or culture medium present in the separated sample may be insufficient to interfere with identification or characterization of the microorganism, or further growth of the microorganism. In some embodiments, the separated sample is 99% pure of contaminating elements, but it may also be 95% pure, 90% pure, 80% pure, 70% pure, 60% pure, 50% pure, or of a minimum purity that still permits identification of the microorganism in the separated sample via a downstream identification technique.

While reference is made to testing for microorganisms and viruses, the methods presented herein may be used for a wide variety of sample types and a wide variety of nucleic acid testing. Thus, by “sample” is meant an animal; a tissue or organ from an animal; a cell (either within a subject, taken directly from a subject, or a cell maintained in culture or from a cultured cell line); a cell lysate (or lysate fraction) or cell extract; a solution containing one or more molecules derived from a cell, cellular material, or viral material; or other samples containing nucleic acids. A sample may also be any body fluid or excretion (for example, but not limited to, blood, urine, stool, saliva, tears, bile, or cerebrospinal fluid) that may or may not contain host or pathogen cells, cell components, or nucleic acids.

In yet another aspect described in further detail herein, microorganisms from a sample or growth medium can be pelleted and interrogated to characterize and/or identify the microorganism present in the sample. As used herein, the term “pellet” is intended to encompass any sample of microorganisms that has been compressed or deposited into a mass of microorganisms. For example, microorganisms from a sample can be compressed or deposited into a mass at the bottom of a tube by centrifugation, or other known methods in the art. The term includes a collection of microorganisms (and/or components thereof) on the bottom and/or sides of a container following centrifugation. In accordance with this invention, microorganisms may be pelleted away (e.g., as a substantially purified microorganism pellet) from non-microorganism or non-microorganism components that may otherwise interfere with characterization and/or identification.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, mRNA, rRNA, cDNA, gDNA, ssDNA, dsDNA, or any combination thereof.

By “probe,” “primer,” or “oligonucleotide” is meant a single-stranded nucleic acid molecule of defined sequence that can base-pair to a second nucleic acid molecule that contains a complementary sequence (the “target”). The stability of the resulting hybrid depends upon the length, GC content, and the extent of the base-pairing that occurs. The extent of base-pairing is affected by parameters such as the degree of complementarity between the probe and target molecules and the degree of stringency of the hybridization conditions. The degree of hybridization stringency is affected by parameters such as temperature, salt concentration, and the concentration of organic molecules such as formamide, and is determined by methods known to one skilled in the art. Probes, primers, and oligonucleotides may be detectably-labeled, either radioactively, fluorescently, or non-radioactively, by methods well-known to those skilled in the art. dsDNA binding dyes may be used to detect dsDNA. It is understood that a “primer” is specifically configured to be extended by a polymerase, whereas a “probe” or “oligonucleotide” may or may not be so configured.

By “dsDNA binding dyes” is meant dyes that fluoresce differentially when bound to double-stranded DNA than when bound to single-stranded DNA or free in solution, usually by fluorescing more strongly. While reference is made to dsDNA binding dyes, it is understood that any suitable dye may be used herein, with some non-limiting illustrative dyes described in U.S. Pat. No. 7,387,887, herein incorporated by reference. Other signal producing substances may be used for detecting nucleic acid amplification and melting, illustratively enzymes, antibodies, etc., as are known in the art.

By “specifically hybridizes” is meant that a probe, primer, or oligonucleotide recognizes and physically interacts (that is, base-pairs) with a substantially complementary nucleic acid (for example, a sample nucleic acid) under high stringency conditions, and does not substantially base pair with other nucleic acids.

By “high stringency conditions” is meant typically to occur at about a melting temperature (Tm) minus 5° C. (i.e. 5° below the Tm of the probe). Functionally, high stringency conditions are used to identify nucleic acid sequences having at least 80% sequence identity.

By “lysis particles” is meant various particles or beads for the lysis of cells, viruses, spores, and other material that may be present in a sample. Various examples use Zirconium (“Zr”) silicate or ceramic beads, but other lysis particles are known and are within the scope of this term, including glass and sand lysis particles. The term “cell lysis component” may include lysis particles, but may also include other components, such as components for chemical lysis, as are known in the art.

While PCR is the amplification method used in the examples herein, it is understood that any amplification method that uses a primer may be suitable. Such suitable procedures include polymerase chain reaction (PCR); strand displacement amplification (SDA); nucleic acid sequence-based amplification (NASBA); cascade rolling circle amplification (CRCA), loop-mediated isothermal amplification of DNA (LAMP); isothermal and chimeric primer-initiated amplification of nucleic acids (ICAN); target based-helicase dependent amplification (HDA); transcription-mediated amplification (TMA), and the like. Therefore, when the term PCR is used, it should be understood to include other alternative amplification methods. For amplification methods without discrete cycles, reaction time may be used where measurements are made in cycles, doubling time, or crossing point (Cp), and additional reaction time may be added where additional PCR cycles are added in the embodiments described herein. It is understood that protocols may need to be adjusted accordingly.

While various examples herein reference human targets and human pathogens, these examples are illustrative only. Methods, kits, and devices described herein may be used to detect or sequence a wide variety of nucleic acid sequences from a wide variety of samples, including, human, veterinary, industrial, and environmental.

Various embodiments disclosed herein use a self-contained nucleic acid analysis pouch to assay a sample for the presence of various biological substances, illustratively antigens and nucleic acid sequences, illustratively in a single closed system. Such systems, including pouches and instruments for use with the pouches, are disclosed in more detail in U.S. Pat. Nos. 8,394,608; and 8,895,295; and 10,464,060, herein incorporated by reference. However, it is understood that such pouches are illustrative only, and the nucleic acid preparation and amplification reactions discussed herein may be performed in any of a variety of open or closed system sample vessels as are known in the art, including 96-well plates, plates of other configurations, arrays, carousels, and the like, using a variety of nucleic acid purification and amplification systems, as are known in the art. While the terms “sample well”, “sample container”, “amplification well”, “amplification container”, or the like are used herein, these terms are meant to encompass blisters wells. tubes, and various other reaction containers, as are used in these amplification systems. In one embodiment, the pouch is used to assay for multiple pathogens. The pouch may include one or more blisters used as sample wells, illustratively in a closed system. Illustratively, various steps may be performed in the optionally disposable pouch, including nucleic acid preparation, primary large volume multiplex PCR, dilution of primary amplification product, and secondary PCR, culminating with optional real-time detection or post-amplification analysis such as melting-curve analysis. Further, it is understood that while the various steps may be performed in pouches of the present invention, one or more of the steps may be omitted for certain uses, and the pouch configuration may be altered accordingly. While many embodiments herein use a multiplex reaction for the first-stage amplification, it is understood that this is illustrative only, and that in some embodiments the first-stage amplification may be singleplex. In one illustrative example, the first-stage singleplex amplification targets housekeeping genes, and the second-stage amplification uses differences in housekeeping genes for identification. Thus, while various embodiments discuss first-stage multiplex amplification, it is understood that this is illustrative only.

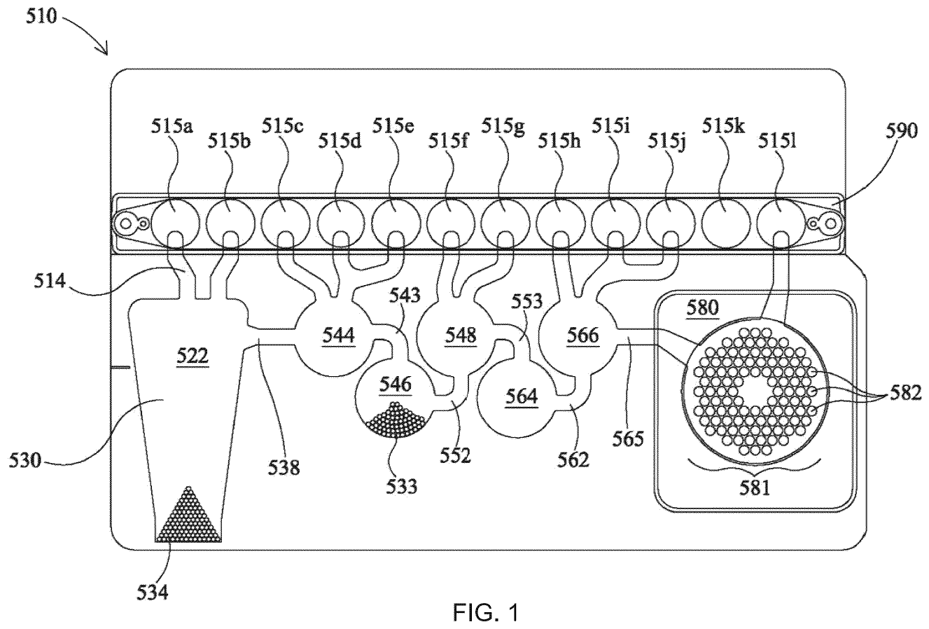

FIG. 1 shows an illustrative pouch 510 that may be used in various embodiments, or may be reconfigured for various embodiments. Pouch 510 is similar to FIG. 15 of U.S. Pat. No. 8,895,295, with like items numbered the same. Fitment 590 is provided with entry channels 515a through 5151, which also serve as reagent reservoirs or waste reservoirs. Illustratively, reagents may be freeze dried in fitment 590 and rehydrated prior to use. Blisters 522, 544, 546, 548, 564, and 566, with their respective channels 514, 538, 543, 552, 553, 562, and 565 are similar to blisters of the same number of FIG. 15 of U.S. Pat. No. 8,895,295. Second-stage reaction zone 580 of FIG. 1 is similar to that of U.S. Pat. No. 8,895,295, but the second-stage wells 582 of high density array 581 are arranged in a somewhat different pattern. The more circular pattern of high density array 581 of FIG. 1 eliminates wells in corners and may result in more uniform filling of second-stage wells 582. As shown, the high density array 581 is provided with 102 second-stage wells 582. Pouch 510 is suitable for use in the Film Array® instrument (BioFire Diagnostics, LLC, Salt Lake City, UT). However, it is understood that the pouch embodiment is illustrative only.

While other containers may be used, illustratively, pouch 510 may be formed of two layers of a flexible plastic film or other flexible material such as polyester, polyethylene terephthalate (PET), polycarbonate, polypropylene, polymethylmethacrylate, mixtures, combinations, and layers thereof that can be made by any process known in the art, including extrusion, plasma deposition, and lamination. For instance, each layer can be composed of one or more layers of material of a single type or more than one type that are laminated together. Metal foils or plastics with aluminum lamination also may be used. Other barrier materials are known in the art that can be sealed together to form the blisters and channels. If plastic film is used, the layers may be bonded together, illustratively by heat sealing. Illustratively, the material has low nucleic acid binding and low protein binding capacity.

For embodiments employing fluorescent monitoring, plastic films that are adequately low in absorbance and auto-fluorescence at the operative wavelengths are preferred. Such material could be identified by testing different plastics, different plasticizers, and composite ratios, as well as different thicknesses of the film. For plastics with aluminum or other foil lamination, the portion of the pouch that is to be read by a fluorescence detection device can be left without the foil. For example, if fluorescence is monitored in second-stage wells 582 of the second-stage reaction zone 580 of pouch 510, then one or both layers at wells 582 would be left without the foil. In the example of PCR, film laminates composed of polyester (Mylar, DuPont, Wilmington DE) of about 0.0048 inch (0.1219 mm) thick and polypropylene films of 0.001-0.003 inch (0.025-0.076 mm) thick perform well. Illustratively, pouch 510 may be made of a clear material capable of transmitting approximately 80%-90% of incident light.

In the illustrative embodiment, the materials are moved between blisters by the application of pressure, illustratively pneumatic pressure, upon the blisters and channels. Accordingly, in embodiments employing pressure, the pouch material illustratively is flexible enough to allow the pressure to have the desired effect. The term “flexible” is herein used to describe a physical characteristic of the material of the pouch. The term “flexible” is herein defined as readily deformable by the levels of pressure used herein without cracking, breaking, crazing, or the like. For example, thin plastic sheets, such as Saran™ wrap and Ziploc® bags, as well as thin metal foil, such as aluminum foil, are flexible. However, only certain regions of the blisters and channels need be flexible, even in embodiments employing pneumatic pressure. Further, only one side of the blisters and channels need to be flexible, as long as the blisters and channels are readily deformable. Other regions of the pouch 510 may be made of a rigid material or may be reinforced with a rigid material. Thus, it is understood that when the terms “flexible pouch” or “flexible sample container” or the like are used, only portions of the pouch or sample container need be flexible.

Illustratively, a plastic film may be used for pouch 510. A sheet of metal, illustratively aluminum, or other suitable material, may be milled or otherwise cut, to create a die having a pattern of raised surfaces. When fitted into a pneumatic press (illustratively A-5302-PDS, Janesville Tool Inc., Milton WI), illustratively regulated at an operating temperature of 195° C., the pneumatic press works like a printing press, melting the sealing surfaces of plastic film only where the die contacts the film. Likewise, the plastic film(s) used for pouch 510 may be cut and welded together using a laser cutting and welding device. Various components, such as PCR primers (illustratively spotted onto the film and dried), antigen binding substrates, magnetic beads, and zirconium silicate beads may be sealed inside various blisters as the pouch 510 is formed. Reagents for sample processing can be spotted onto the film prior to sealing, either collectively or separately. In one embodiment, nucleotide tri-phosphates (NTPs) are spotted onto the film separately from polymerase and primers, essentially eliminating activity of the polymerase until the reaction may be hydrated by an aqueous sample. If the aqueous sample has been heated prior to hydration, this creates the conditions for a true hot-start PCR and reduces or eliminates the need for expensive chemical hot-start components. In another embodiment, components may be provided in powder or pill form and are placed into blisters prior to final sealing.

Pouch 510 may be used in a manner similar to that described in U.S. Pat. No. 8,895,295. In one illustrative embodiment, a 300 μl mixture comprising the sample to be tested (100 μl) and lysis buffer (200 μl) may be injected into an injection port (not shown) in fitment 590 near entry channel 515a, and the sample mixture may be drawn into entry channel 515a. Water may also be injected into a second injection port (not shown) of the fitment 590 adjacent entry channel 515l, and is distributed via a channel (not shown) provided in fitment 590, thereby hydrating up to eleven different reagents, each of which were previously provided in dry form at entry channels 515b through 515l. Illustrative methods and devices for injecting sample and hydration fluid (e.g. water or buffer) are disclosed in U.S. Pat. No. 10,464,060, herein incorporated by reference in its entirety, although it is understood that these methods and devices are illustrative only and other ways of introducing sample and hydration fluid into pouch 510 are within the scope of this disclosure. These reagents illustratively may include freeze-dried PCR reagents, DNA extraction reagents, wash solutions, immunoassay reagents, or other chemical entities. Illustratively, the reagents are for nucleic acid extraction, first-stage multiplex PCR, dilution of the multiplex reaction, and preparation of second-stage PCR reagents, as well as control reactions. In the embodiment shown in FIG. 1, all that need be injected is the sample solution in one injection port and water in the other injection port. After injection, the two injection ports may be sealed. For more information on various configurations of pouch 510 and fitment 590, see U.S. Pat. No. 8,895,295, already incorporated by reference.

After injection, the sample may be moved from injection channel 515a to lysis blister 522 via channel 514. Lysis blister 522 is provided with beads or particles 534, such as ceramic beads or other abrasive elements, and is configured for vortexing via impaction using rotating blades or paddles provided within the FilmArray® instrument. Bead-milling, by shaking, vortexing, sonicating, and similar treatment of the sample in the presence of lysis particles such as zirconium silicate (ZS) beads 534, is an effective method to form a lysate. It is understood that, as used herein, terms such as “lyse,” “lysing,” and “lysate” are not limited to rupturing cells, but that such terms include disruption of non-cellular particles, such as viruses. In another embodiment, a paddle beater using reciprocating or alternating paddles, such as those described in US 2019-0344269, herein incorporated by reference in its entirety, may be used for lysis in this embodiment, as well as in the other embodiments described herein.

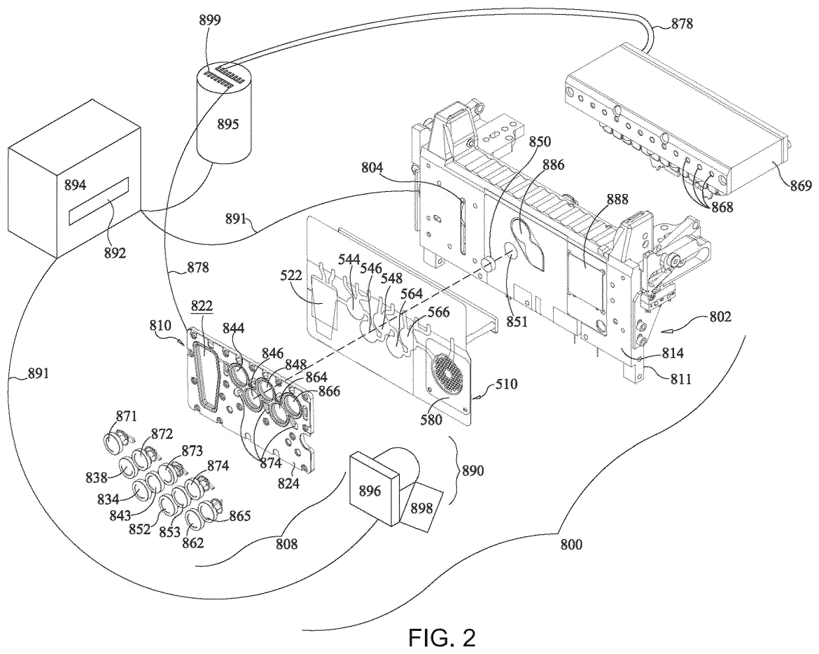

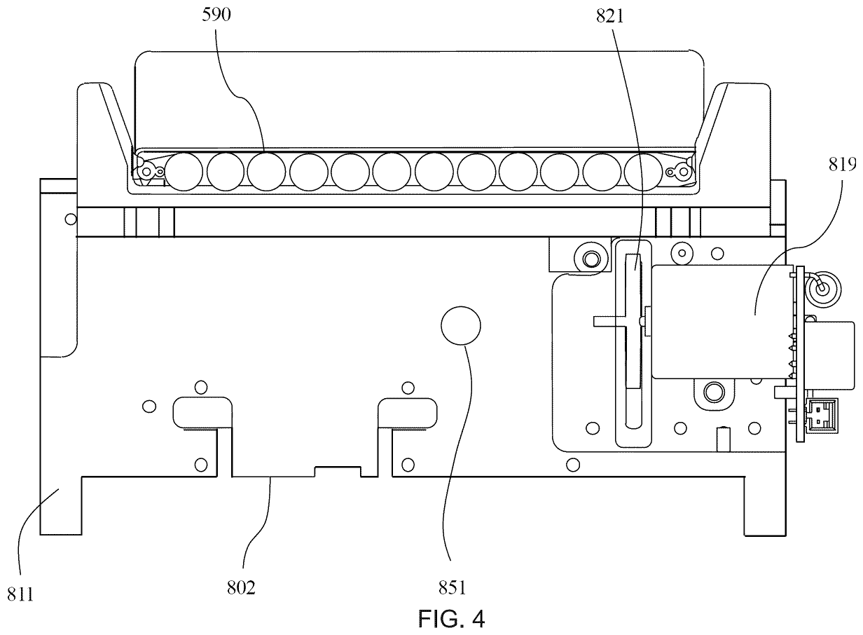

FIG. 4 shows a bead beating motor 819, comprising blades 821 that may be mounted on a first side 811 of support member 802, of instrument 800 shown in FIG. 2. Blades may extend through slot 804 to contact pouch 510. It is understood, however, that motor 819 may be mounted on other structures of instrument 800. In one illustrative embodiment, motor 819 is a Mabuchi RC-280SA-2865 DC Motor (Chiba, Japan), mounted on support member 802. In one illustrative embodiment, the motor is turned at 5,000 to 25,000 rpm, more illustratively 10,000 to 20,000 rpm, and still more illustratively approximately 15,000 to 18,000 rpm. For the Mabuchi motor, it has been found that 7.2V provides sufficient rpm for lysis. It is understood, however, that the actual speed may be somewhat slower when the blades 821 are impacting pouch 510. Other voltages and speeds may be used for lysis depending on the motor and paddles used. Optionally, controlled small volumes of air may be provided into the bladder 822 adjacent lysis blister 522. It has been found that in some embodiments, partially filling the adjacent bladder with one or more small volumes of air aids in positioning and supporting lysis blister during the lysis process. Alternatively, another structure, illustratively a rigid or compliant gasket or other retaining structure around lysis blister 522, can be used to restrain pouch 510 during lysis. It is also understood that motor 819 is illustrative only, and other devices may be used for milling, shaking, or vortexing the sample. In some embodiments, chemicals or heat may be used in addition to or instead of mechanical lysis.

Once the sample material has been adequately lysed, the sample is moved to a nucleic acid extraction zone, illustratively through channel 538, blister 544, and channel 543, to blister 546, where the sample is mixed with a nucleic acid-binding substance, such as silica-coated magnetic beads 533. Alternatively, magnetic beads 533 may be rehydrated, illustratively using fluid provided from one of the entry channel 515c-515e, and then moved through channel 543 to blister 544, and then through channel 538 to blister 522. The mixture is allowed to incubate for an appropriate length of time, illustratively approximately 10 seconds to 10 minutes. A retractable magnet located within the instrument adjacent blister 546 captures the magnetic beads 533 from the solution, forming a pellet against the interior surface of blister 546. If incubation takes place in blister 522, multiple portions of the solution may need to be moved to blister 546 for capture. The liquid is then moved out of blister 546 and back through blister 544 and into blister 522, which is now used as a waste receptacle. One or more wash buffers from one or more of injection channels 515c to 515e are provided via blister 544 and channel 543 to blister 546. Optionally, the magnet is retracted and the magnetic beads 533 are washed by moving the beads back and forth from blisters 544 and 546 via channel 543. Once the magnetic beads 533 are washed, the magnetic beads 533 are recaptured in blister 546 by activation of the magnet, and the wash solution is then moved to blister 522. This process may be repeated as necessary to wash the lysis buffer and sample debris from the nucleic acid-binding magnetic beads 533.

After washing, elution buffer stored at injection channel 515f is moved to blister 548, and the magnet is retracted. The solution is cycled between blisters 546 and 548 via channel 552, breaking up the pellet of magnetic beads 533 in blister 546 and allowing the captured nucleic acids to dissociate from the beads and come into solution. The magnet is once again activated, capturing the magnetic beads 533 in blister 546, and the eluted nucleic acid solution is moved into blister 548.

First-stage PCR master mix from injection channel 515g is mixed with the nucleic acid sample in blister 548. Optionally, the mixture is mixed by forcing the mixture between 548 and 564 via channel 553. After several cycles of mixing, the solution is contained in blister 564, where a pellet of first-stage PCR primers is provided, at least one set of primers for each target, and first-stage multiplex PCR is performed. If RNA targets are present, a reverse transcription (RT) step may be performed prior to or simultaneously with the first-stage multiplex PCR. First-stage multiplex PCR temperature cycling in the FilmArray® instrument is illustratively performed for 15-20 cycles, although other levels of amplification may be desirable, depending on the requirements of the specific application. The first-stage PCR master mix may be any of various master mixes, as are known in the art. In one illustrative example, the first-stage PCR master mix may be any of the chemistries disclosed in U.S. Pat. No. 9,932,634, herein incorporated by reference, for use with PCR protocols taking 20 seconds or less per cycle.

After first-stage PCR has proceeded for the desired number of cycles, the sample may be diluted, illustratively by forcing most of the sample back into blister 548, leaving only a small amount in blister 564, and adding second-stage PCR master mix from injection channel 515i. Alternatively, a dilution buffer from 515i may be moved to blister 566 then mixed with the amplified sample in blister 564 by moving the fluids back and forth between blisters 564 and 566. If desired, dilution may be repeated several times, using dilution buffer from injection channels 515j and 515k, or injection channel 515k may be reserved, illustratively, for sequencing or for other post-PCR analysis, and then adding second-stage PCR master mix from injection channel 515h to some or all of the diluted amplified sample. It is understood that the level of dilution may be adjusted by altering the number of dilution steps or by altering the percentage of the sample discarded prior to mixing with the dilution buffer or second-stage PCR master mix comprising components for amplification, illustratively a polymerase, dNTPs, and a suitable buffer, although other components may be suitable, particularly for non-PCR amplification methods. If desired, this mixture of the sample and second-stage PCR master mix may be pre-heated in blister 564 prior to movement to second-stage wells 582 for second-stage amplification. Such preheating may obviate the need for a hot-start component (antibody, chemical, or otherwise) in the second-stage PCR mixture.

In one embodiment, the illustrative second-stage PCR master mix is incomplete, lacking primer pairs, and each of the 102 second-stage wells 582 is pre-loaded with a specific PCR primer pair. In other embodiments, the master mix may lack other components (e.g., polymerase, Mg²⁺, etc.) and the lacking components may be pre-loaded in the array. If desired, second-stage PCR master mix may lack other reaction components, and these components may be pre-loaded in the second-stage wells 582 as well. Each primer pair may be similar to or identical to a first-stage PCR primer pair or may be nested within the first-stage primer pair. Movement of the sample from blister 564 to the second-stage wells 582 completes the PCR reaction mixture. Once high density array 581 is filled, the individual second-stage reactions are sealed in their respective second-stage blisters by any number of means, as is known in the art. Illustrative ways of filling and sealing the high density array 581 without cross-contamination are discussed in U.S. Pat. No. 8,895,295, already incorporated by reference. Illustratively, the various reactions in wells 582 of high density array 581 are simultaneously or individually thermal cycled, illustratively with one or more Peltier devices, although other means for thermal cycling are known in the art.

In certain embodiments, second-stage PCR master mix contains the dsDNA binding dye LCGreen® Plus (BioFire Diagnostics, LLC) to generate a signal indicative of amplification. However, it is understood that this dye is illustrative only, and that other signals may be used, including other dsDNA binding dyes and probes that are labeled fluorescently, radioactively, chemiluminescently, enzymatically, or the like, as are known in the art. Alternatively, wells 582 of array 581 may be provided without a signal, with results reported through subsequent processing.

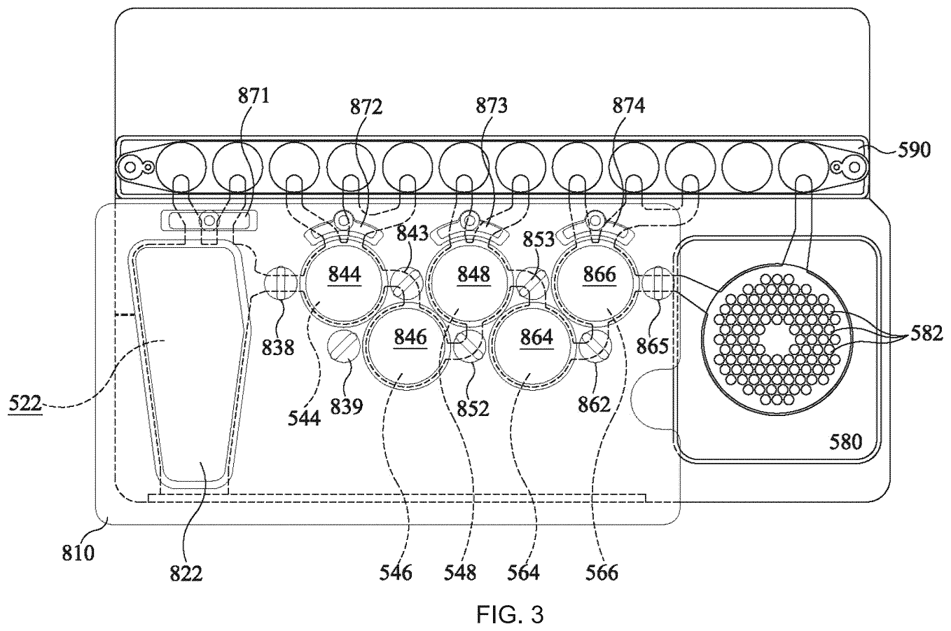

When pneumatic pressure is used to move materials within pouch 510, in one embodiment, a “bladder” may be employed. The bladder assembly 810, a portion of which is shown in FIGS. 2-3, includes a bladder plate 824 housing a plurality of inflatable bladders 822, 844, 846, 848, 864, and 866, each of which may be individually inflatable, illustratively by a compressed gas source. Because the bladder assembly 810 may be subjected to compressed gas and used multiple times, the bladder assembly 810 may be made from tougher or thicker material than the pouch. Alternatively, bladders 822, 844, 846, 848, 864, and 866 may be formed from a series of plates fastened together with gaskets, seals, valves, and pistons. Other arrangements are within the scope of this invention. Alternatively, an array or mechanical actuators and seals may be used to seal channels and direct movement of fluids between blisters. A system of mechanical seals and actuators that may be adapted for the instruments described herein is described in detail in US 2019-0344269, the entirety of which is already incorporated by reference.

Success of the secondary PCR reactions is dependent upon template generated by the multiplex first-stage reaction. Typically, PCR is performed using DNA of high purity. Methods such as phenol extraction or commercial DNA extraction kits provide DNA of high purity. Samples processed through the pouch 510 may require accommodations be made to compensate for a less pure preparation. PCR may be inhibited by components of biological samples, which is a potential obstacle. Illustratively, hot-start PCR, higher concentration of Taq polymerase enzyme, adjustments in MgCl₂ concentration, adjustments in primer concentration, addition of engineered enzymes that are resistant to inhibitors, and addition of adjuvants (such as DMSO, TMSO, or glycerol) optionally may be used to compensate for lower nucleic acid purity. While purity issues are likely to be more of a concern with first-stage amplification, it is understood that similar adjustments may be provided in the second-stage amplification as well.

When pouch 510 is placed within the instrument 800, the bladder assembly 810 is pressed against one face of the pouch 510, so that if a particular bladder is inflated, the pressure will force the liquid out of the corresponding blister in the pouch 510. In addition to bladders corresponding to many of the blisters of pouch 510, the bladder assembly 810 may have additional pneumatic actuators, such as bladders or pneumatically-driven pistons, corresponding to various channels of pouch 510. FIGS. 2-3 show an illustrative plurality of pistons or hard seals 838, 843, 852, 853, and 865 that correspond to channels 538, 543, 553, and 565 of pouch 510, as well as seals 871, 872, 873, 874 that minimize backflow into fitment 590. When activated, hard seals 838, 843, 852, 853, and 865 form pinch valves to pinch off and close the corresponding channels. To confine liquid within a particular blister of pouch 510, the hard seals are activated over the channels leading to and from the blister, such that the actuators function as pinch valves to pinch the channels shut. Illustratively, to mix two volumes of liquid in different blisters, the pinch valve actuator sealing the connecting channel is activated, and the pneumatic bladders over the blisters are alternately pressurized, forcing the liquid back and forth through the channel connecting the blisters to mix the liquid therein. The pinch valve actuators may be of various shapes and sizes and may be configured to pinch off more than one channel at a time. While pneumatic actuators are discussed herein, it is understood that other ways of providing pressure to the pouch are contemplated, including various electromechanical actuators such as linear stepper motors, motor-driven cams, rigid paddles driven by pneumatic, hydraulic or electromagnetic forces, rollers, rocker-arms, and in some cases, cocked springs. In addition, there are a variety of methods of reversibly or irreversibly closing channels in addition to applying pressure normal to the axis of the channel. These include kinking the bag across the channel, heat-sealing, rolling an actuator, and a variety of physical valves sealed into the channel such as butterfly valves and ball valves. Additionally, small Peltier devices or other temperature regulators may be placed adjacent the channels and set at a temperature sufficient to freeze the fluid, effectively forming a seal. Also, while the design of FIG. 1 is adapted for an automated instrument featuring actuator elements positioned over each of the blisters and channels, it is also contemplated that the actuators could remain stationary, and the pouch 510 could be transitioned such that a small number of actuators could be used for several of the processing stations including sample disruption, nucleic-acid capture, first and second-stage PCR, and processing stations for other applications of the pouch 510 such as immuno-assay and immuno-PCR. Rollers acting on channels and blisters could prove particularly useful in a configuration in which the pouch 510 is translated between stations. Thus, while pneumatic actuators are used in the presently disclosed embodiments, when the term “pneumatic actuator” is used herein, it is understood that other actuators and other ways of providing pressure may be used, depending on the configuration of the pouch and the instrument.

Turning back to FIG. 2, each pneumatic actuator is connected to compressed air source 895 via valves 899. While only several hoses 878 are shown in FIG. 2, it is understood that each pneumatic fitting is connected via a hose 878 to the compressed gas source 895. Compressed gas source 895 may be a compressor, or, alternatively, compressed gas source 895 may be a compressed gas cylinder, such as a carbon dioxide cylinder. Compressed gas cylinders are particularly useful if portability is desired. Other sources of compressed gas are within the scope of this invention. Similar pneumatic control may be provided, for example, for control of fluid movement in the pouches described herein, or other actuators, servos, or the like may be provided.

Several other components of the instrument are also connected to compressed gas source 895. A magnet 850, which is mounted on a second side 814 of support member 802, is illustratively deployed and retracted using gas from compressed gas source 895 via hose 878, although other methods of moving magnet 850 are known in the art. Magnet 850 sits in recess 851 in support member 802. It is understood that recess 851 can be a passageway through support member 802, so that magnet 850 can contact blister 546 of pouch 510. However, depending on the material of support member 802, it is understood that recess 851 need not extend all the way through support member 802, as long as when magnet 850 is deployed, magnet 850 is close enough to provide a sufficient magnetic field at blister 546, and when magnet 850 is fully retracted, magnet 850 does not significantly affect any magnetic beads 533 present in blister 546. While reference is made to retracting magnet 850, it is understood that an electromagnet may be used and the electromagnet may be activated and inactivated by controlling flow of electricity through the electromagnet. Thus, while this specification discusses withdrawing or retracting the magnet, it is understood that these terms are broad enough to incorporate other ways of withdrawing the magnetic field. It is understood that the pneumatic connections may be pneumatic hoses or pneumatic air manifolds, thus reducing the number of hoses or valves required. It is understood that similar magnets and methods for activating the magnets may be used in other embodiments.

The various pneumatic pistons 868 of pneumatic piston array 869 are also connected to compressed gas source 895 via hoses 878. While only two hoses 878 are shown connecting pneumatic pistons 868 to compressed gas source 895, it is understood that each of the pneumatic pistons 868 are connected to compressed gas source 895. Twelve pneumatic pistons 868 are shown.

A pair of temperature control elements are mounted on a second side 814 of support member 802. As used herein, the term “temperature control element” refers to a device that adds heat to or removes heat from a sample. Illustrative examples of a temperature control element include, but are not limited to, heaters, coolers, Peltier devices, resistive heaters, induction heaters, electromagnetic heaters, thin film heaters, printed element heaters, positive temperature coefficient heaters, and combinations thereof. A temperature control element may include multiple heaters, coolers, Peltiers, etc. In one aspect, a given temperature control element may include more than one type of heater or cooler. For instance, an illustrative example of a temperature control element may include a Peltier device with a separate resistive heater applied to the top and/or the bottom face of the Peltier. While the term “heater” is used throughout the specification, it is understood that other temperature control elements may be used to adjust the temperature of the sample.

As discussed above, first-stage heater 886 may be positioned to heat and cool the contents of blister 564 for first-stage PCR. As seen in FIG. 2, second-stage heater 888 may be positioned to heat and cool the contents of second-stage blisters of array 581 of pouch 510, for second-stage PCR. It is understood, however, that these heaters could also be used for other heating purposes, and that other heaters may be included, as appropriate for the particular application.

As discussed above, while Peltier devices, which thermocycle between two or more temperatures, are effective for PCR, it may be desirable in some embodiments to maintain heaters at a constant temperature. Illustratively, this can be used to reduce run time, by eliminating time needed to transition the heater temperature beyond the time needed to transition the sample temperature. Also, such an arrangement can improve the electrical efficiency of the system as it is only necessary to thermally cycle the smaller sample and sample vessel, not the much larger (more thermal mass) Peltier devices. For instance, an instrument may include multiple heaters (i.e., two or more) at temperatures set for, for example, annealing, extension, denaturation that are positioned relative to the pouch to accomplish thermal cycling. Two heaters may be sufficient for many applications. In various embodiments, the heaters can be moved, the pouch can be moved, or fluids can be moved relative to the heaters to accomplish thermal cycling. Illustratively, the heaters may be arranged linearly, in a circular arrangement, or the like. Types of suitable heaters have been discussed above, with reference to first-stage PCR.

When fluorescent detection is desired, an optical array 890 may be provided. As shown in FIG. 2, optical array 890 includes a light source 898, illustratively a filtered LED light source, filtered white light, or laser illumination, and a camera 896. Camera 896 illustratively has a plurality of photodetectors each corresponding to a second-stage well 582 in pouch 510. Alternatively, camera 896 may take images that contain all of the second-stage wells 582, and the image may be divided into separate fields corresponding to each of the second-stage wells 582. Depending on the configuration, optical array 890 may be stationary, or optical array 890 may be placed on movers attached to one or more motors and moved to obtain signals from each individual second-stage well 582. It is understood that other arrangements are possible. Some embodiments for second-stage heaters provide the heaters on the opposite side of pouch 510 from that shown in FIG. 2. Such orientation is illustrative only and may be determined by spatial constraints within the instrument. Provided that second-stage reaction zone 580 is provided in an optically transparent material, photodetectors and heaters may be on either side of array 581.

As shown, a computer 894 controls valves 899 of compressed air source 895, and thus controls all of the pneumatics of instrument 800. In addition, many of the pneumatic systems in the instrument may be replaced with mechanical actuators, pressure applying means, and the like in other embodiments. Computer 894 also controls heaters 886 and 888, and optical array 890. Each of these components is connected electrically, illustratively via cables 891, although other physical or wireless connections are within the scope of this invention. It is understood that computer 894 may be housed within instrument 800 or may be external to instrument 800. Further, computer 894 may include built-in circuit boards that control some or all of the components, and may also include an external computer, such as a desktop or laptop PC, to receive and display data from the optical array. An interface, illustratively a keyboard interface, may be provided including keys for inputting information and variables such as temperatures, cycle times, etc. Illustratively, a display 892 is also provided. Display 892 may be an LED, LCD, or other such display, for example.

Other instruments known in the art teach PCR within a sealed flexible container. See, e.g., U.S. Pat. Nos. 6,645,758, 6,780,617, and 9,586,208, herein incorporated by reference. However, including the cell lysis within the sealed PCR vessel can improve ease of use and safety, particularly if the sample to be tested may contain a biohazard. In the embodiments illustrated herein, the waste from cell lysis, as well as that from all other steps, remains within the sealed pouch. Still, it is understood that the pouch contents could be removed for further testing.

Turning back to FIG. 2, instrument 800 includes a support member 802 that could form a wall of a casing or be mounted within a casing. Instrument 800 may also include a second support member (not shown) that is optionally movable with respect to support member 802, to allow insertion and withdrawal of pouch 510. Illustratively, a lid may cover pouch 510 once pouch 510 has been inserted into instrument 800. In another embodiment, both support members may be fixed, with pouch 510 held into place by other mechanical means or by pneumatic pressure.

In the illustrative example, heaters 886 and 888 are mounted on support member 802. However, it is understood that this arrangement is illustrative only and that other arrangements are possible. Illustrative heaters include Peltiers and other block heaters, resistive heaters, electromagnetic heaters, and thin film heaters, as are known in the art, to thermocycle the contents of blister 864 and second-stage reaction zone 580. Bladder plate 810, with bladders 822, 844, 846, 848, 864, 866, hard seals 838, 843, 852, 853, and seals 871, 872, 873, 874 form bladder assembly 808, which may illustratively be mounted on a moveable support structure that may be moved toward pouch 510, such that the pneumatic actuators are placed in contact with pouch 510. When pouch 510 is inserted into instrument 800 and the movable support member is moved toward support member 802, the various blisters of pouch 510 are in a position adjacent to the various bladders of bladder assembly 810 and the various seals of assembly 808, such that activation of the pneumatic actuators may force liquid from one or more of the blisters of pouch 510 or may form pinch valves with one or more channels of pouch 510. The relationship between the blisters and channels of pouch 510 and the bladders and seals of assembly 808 is illustrated in more detail in FIG. 3.

### Methods of Cell Lysis and Nucleic Acid Recovery

Disclosed herein are methods and systems for preparing a nucleic acid sample. The methods and systems described herein are designed for rapid preparation of a nucleic acid sample. As will be explained in greater detail herein below, the methods and systems described herein are able to capitalize on the kinetic efficiency of certain portions of sample preparation (i.e., sample lysis, recovery of nucleic acids from the lysate with a medium such as silica coated magnetic particles, washing the recovery media, and elution of the nucleic acids from the recovery media) to shorten sample preparation as much as possible while still providing high-quality extracted nucleic acids for downstream amplification or other analyses. The methods described herein include focus on one or more of fast mechanical lysis, preferably lysis is conducted in the presence of silica-coated magnetic particles, fast recovery of silica-coated magnetic particles from the lysate, rapid washing of the magnetic particles, and fast, efficient elution of captured nucleic acids from the silica-coated magnetic particles. The methods and systems described herein are also designed for rapid preparation of a nucleic acid sample with buffer compositions that are aqueous and free of alcohols or organic solvents. Illustratively, one or more of the buffer compositions used in the methods described herein suitably may be provided in a sample preparation device as ready to use, shelf-stable liquids that can be stored under ambient conditions and/or provided as a ready to use dried powder compositions that can be rehydrated with a rehydration fluid (e.g., water) for use while performing the steps of the method. Successful sample preparation (i.e., lysis, recovery of nucleic acids from the lysate, washing, and elution) is important for maximizing the sensitivity of molecular assays while ensuring reliable, consistent results. In particular, PCR fidelity and efficiency depend on the purity and concentration of the nucleic acid template added to a reaction. As time-to-result in molecular assays is ever shortened, reducing the time in sample preparation is important for reducing overall assay time. The methods and systems described herein greatly reduce the time needed for sample preparation with mechanical lysis without sacrificing the integrity, purity, and concentration of the input nucleic acid template to ensure reliable, consistent results.

One exemplary method of preparing a nucleic acid sample includes steps of providing a sample container comprising a first chamber, providing a sample (e.g., a nasal swab, a saliva sample, a sputum sample, blood, urine, etc.) suspected of containing one or more target nucleic acids (e.g., target nucleic acids in one or more microorganisms) and a sample buffer comprising a buffering agent, a chaotropic salt, and a non-ionic surfactant. The sample and sample buffer (a first mixture) are combined with a quantity of lysis particles (e.g., zirconium silicate) and a first quantity of magnetic silica particles. The sample, sample buffer, lysis particles, and the magnetic particles may be disposed in the first chamber and suitably may be agitated (e.g., bead beaten) in the first chamber for a period of time sufficient to produce a lysate. In various embodiments, the mixture comprising the sample, sample buffer, lysis particles, and the magnetic particles may be combined and then disposed in the first chamber, the first chamber may comprise one or more components (e.g., the lysis particles and/or the magnetic particles), the sample and buffer may be added to a first chamber that includes the lysis particles and the magnetic particles may be added after, or any conceivable combination thereof. The sample, buffer, lysis particles, and magnetic particles may be agitated using any method or device known in the art for producing a lysate. The Roche MagnaLyser is an example of a commercial bead beater instrument that can be used to agitate a container containing such a mixture to produce a lysate. The bead beater discussed herein above with reference to FIG. 4 is another example of a bead beater instrument that suitably may be used for bead beating in the methods described herein.

After bead beating to produce a lysate, a second quantity of nucleic acid binding magnetic particles may be mixed into the lysate. After mixing the second quantity of nucleic acid binding magnetic particles into the lysate and, optionally, incubating the magnetic particles in the lysate for a period of time (e.g., 5 seconds to 2 minutes), the first and second quantities of nucleic acid binding magnetic particles may be recovered from the lysate-illustratively with a magnet. The Pickpen is an example of a device that can be used to recover magnetic particles from a solution. Magnet 850 discussed with reference to FIG. 2 is another example of a magnet that can be used to recover magnetic particles from a lysate. After recovery, the magnetic particles may be transferred to another container, released, and washed one or more times to remove lysate residue. The magnetic particles may then be recaptured (illustratively with the magnet) so that the wash buffer can be removed, the magnetic particles may be released again and mixed with an elution buffer to elute the captured nucleic acids from the magnetic particles. The magnetic particles may be captured again and the elution buffer may be recovered and transferred to a clean tube. Alternatively, the magnet may be used to remove the magnetic particles from the tube and the magnetic particles may be disposed of. In any case, the recovered nucleic acids in the elution buffer may be used for a variety of downstream assays, such as, but not limited to, assaying the elution buffer for presence of the one or more target nucleic acids suspected to be in the sample with one or more of nucleic acid amplification and detection, sequencing, next generation sequencing, and the like. That is, if the nucleic acids in the sample are from cells or viruses (e.g., pathogen cells), the assay may be used to identify the cells or viruses that the nucleic acids are derived from.

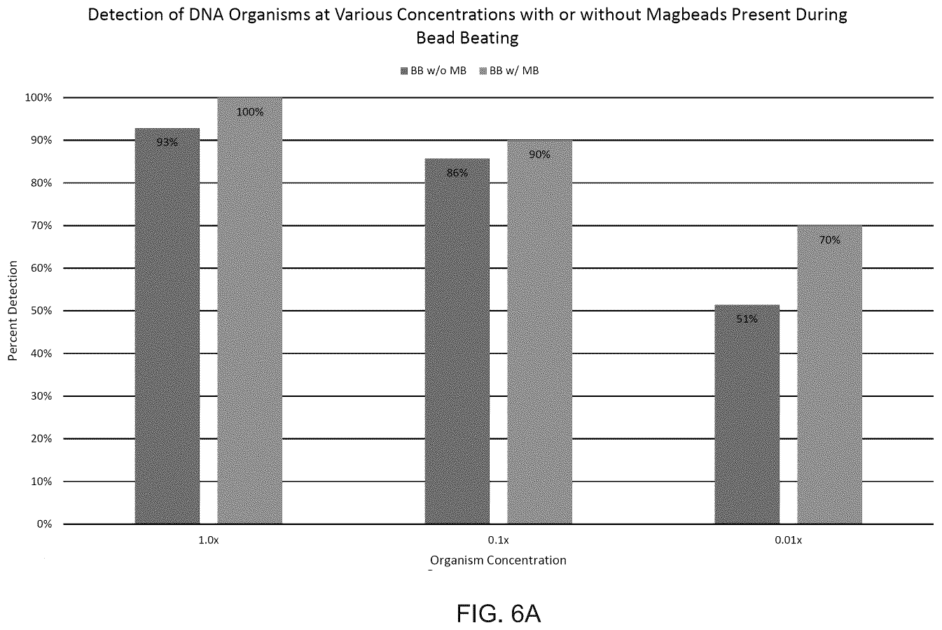

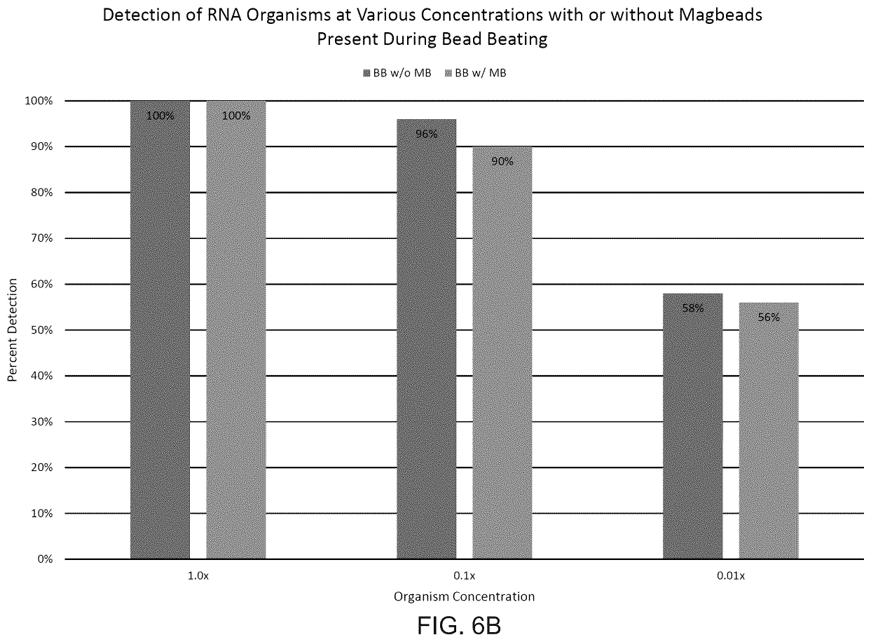

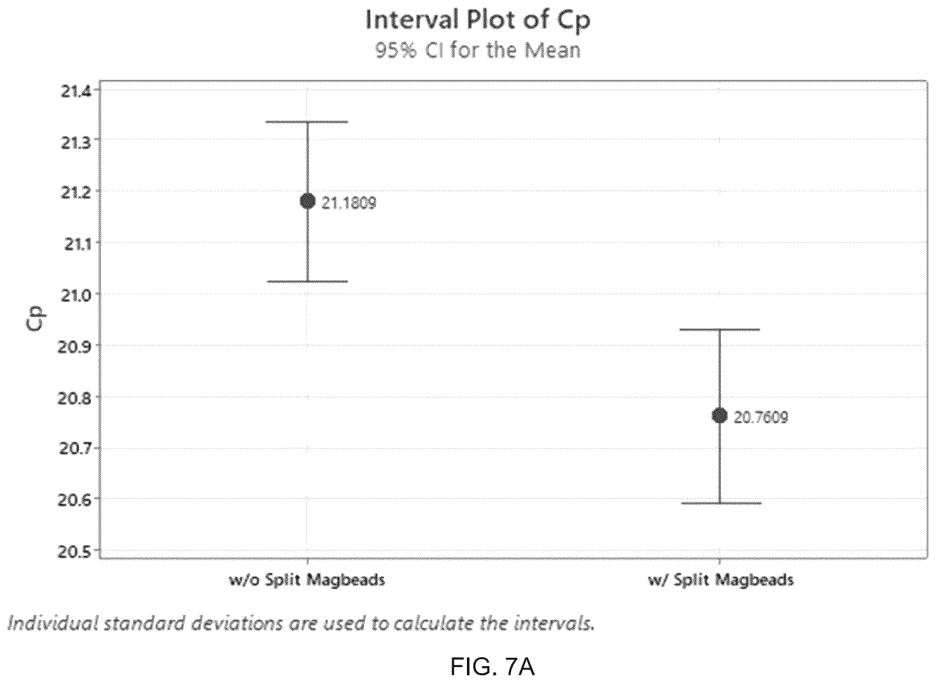

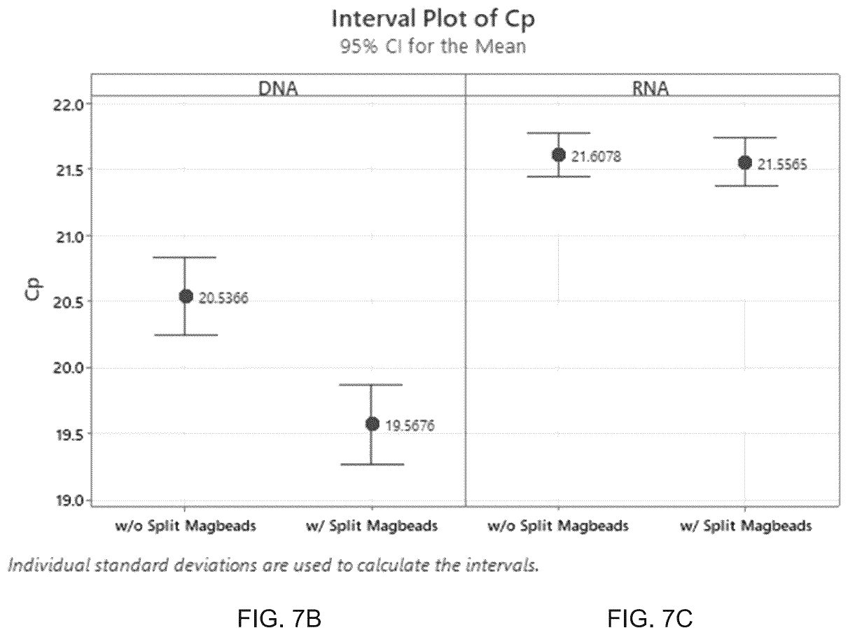

It has been found by the inventors in the present case that lysis and nucleic acid recovery are more efficient when lysis is performed in the presence of magnetic silica particles. This is illustrated, for example, in FIGS. 6A-7C. FIGS. 6A and 6B are bar graphs illustrating precent detection of organisms in sets of assays based on amplification from DNA (FIG. 6A) and sets of assays based on amplification from RNA (FIG. 6B) at various concentrations with (BB w/MB) or without (BB w/o MB) silica-coated magnetic particles present during bead beating lysis. FIG. 6A shows that percent detection for DNA-based assays is consistently better at 1×LOD, 0.1×LOD, and 0.01×LOD when bead beating is performed in the presence of silica-coated magnetic particles. RNA-based assays, however, appear to be about equivalent when silica-coated magnetic particles are added after lysis or when bead beating is performed in the presence of silica-coated magnetic particles. FIG. 7A illustrates average crossing points (Cps) for mixed DNA and RNA assays where a first quantity of magnetic particles were added to the sample prior to forming the lysate and a second quantity of magnetic particles were added after lysis (w/ Split Magbeads) as compared to control assays where magnetic particles are added after lysis (w/o Split Magbeads). An earlier Cp (as compared to the control) means that fewer cycles of amplification were needed in order to detect amplification. Generally, an earlier Cp earlier than the control means that the nucleic acids added into the reaction in the ‘w/Split Magbeads’ case were more concentrated than the nucleic acids obtained when the magnetic particles were added after lysis. Assuming 100% efficiency of template amplification, one cycle of Cp improvement represents about a 2-fold increase in input concentration of template nucleic acid, a two cycle Cp improvement represents about a 4-fold increase, a three cycle Cp improvement represents about a 8-fold increase, etc. (by the general formula, an n cycle Cp improvement represents about a 2ⁿ-fold increase in input concentration of target cells or template nucleic acid). Data in FIG. 7A show that the average Cp for mixed DNA and RNA assays where a first quantity of magnetic particles were added to the sample prior to forming the lysate and a second quantity of magnetic particles were added after lysis (w/Split Magbeads) was better than the control where the magnetic particles were added after forming the lysate. The improvement in Cp demonstrates that protocols ‘w/Split Magbeads’ improved by about 0.42 cycles of Cp, which represents an average of about a 1.34-fold increase in nucleic acid concentration when the magnetic particles were included in the lysis, as compared to controls where magnetic particles were added after lysis was completed. FIGS. 7B and 7C illustrate average Cp data for DNA-dependent assays (FIG. 7B) and RNA-dependent assays (FIG. 7C). FIG. 7B illustrates that DNA-dependent assays showed about a 1 Cp improvement, which represents an overall increase in DNA recovery from a lysate of about 2-fold when a first quantity of magnetic particles were added prior to formation of the lysate and a second quantity of magnetic particles were added after lysis. FIG. 7C illustrates that Cp performance for RNA recovered from magnetic particles present during lysis was about equivalent to the control. Taken together, these data demonstrate that nucleic acids (i.e., DNA and RNA) recovered when a first quantity of magnetic particles were included during lysis formation and a second quantity of magnetic particles were added after lysis formation are more concentrated and likely of higher quality as compared to nucleic acids recovered when silica-coated magnetic particles are added after lysis. In addition, while the Cp changes may not represent a large change in the number of cycles needed to detect a given nucleic acid, the greater concentration of nucleic acids obtained when magnetic particles are included in lysis likely means that fewer missed detections may occur with organisms at or near the limit of detection. This is reflected in FIGS. 6A and 6B.

These data are surprising and unexpected because including magnetic silica particles in a mechanical lysis has traditionally been disfavored by persons of ordinary skill in the art. It has long been believed that having silica-coated magnetic particles present during mechanical lysis could damage the magnetic particles by breaking the silica coating off the particles. It was believed that loss of silica from the silica-coated magnetic particles could reduce the nucleic acid binding capacity of the magnetic particles, and, in addition, the nucleic acids could be lost by binding to the unrecoverable bits of silica broken off the magnetic particles. Nevertheless, when organisms are mechanically lysed (e.g., by bead beating) in the presence of magnetic silica particles, nucleic acid binding and recovery appears to be better. Nucleic acid binding to the magnetic silica particles may occur during lysis and nucleic acid binding may be more efficient and recovery may be greater. In fact, at least for DNA, the nucleic acid binding and recovery of nucleic acids appears to be significantly better. RNA assays are not necessarily helped by including magnetic particles in the lysis, but they are not hurt. It also appears that the quality of the recovered nucleic acid is better when the magnetic silica particles are included in the lysis. For example, it is believed that there may be fewer proteins and other inhibitors co-isolated with the nucleic acids and the nucleic acids may be more concentrated. This is evidenced by the fact that fewer washes of the magnetic silica particles are needed when the magnetic silica particles are included in the lysis.

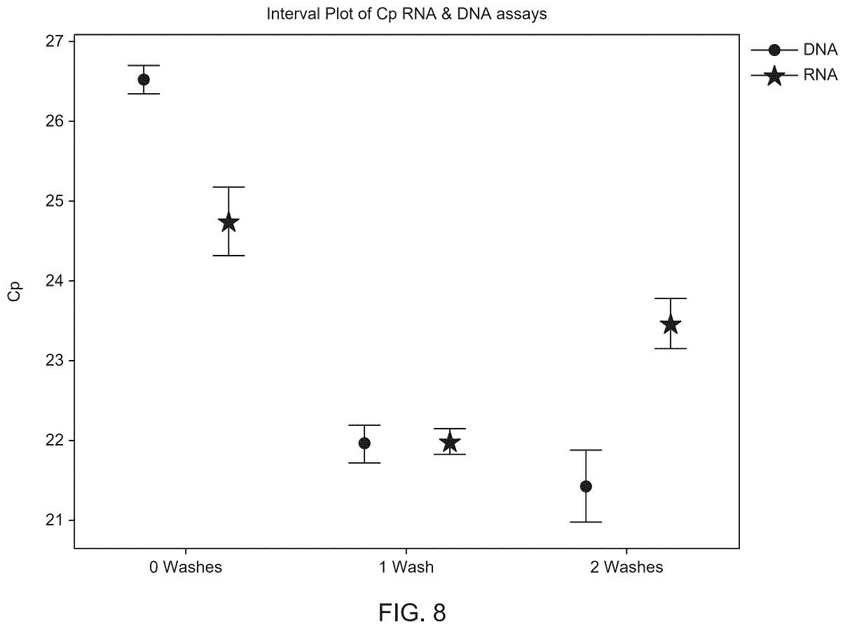

Washing differences are illustrated in FIG. 8, which compares the Cps for amplification of nucleic acids recovered from magnetic particles included in the lysis with no washes, one wash, and two washes; except for the number of washes, the samples were identical. The no wash condition was clearly detrimental for both DNA and RNA assays, as compared to the one wash condition. This is likely due to the presence of PCR inhibitors co-isolated with the nucleic acids in the no wash condition. The one wash condition had improved Cps for both RNA and DNA assays, suggesting that one wash is sufficient to remove most PCR inhibitors without reducing the amount of nucleic acids that can be recovered from the magnetic particles. The two-wash condition appears that it may be slightly better for DNA assays and slightly worse for RNA assays, suggesting that some RNA may have been washed away. In any case, the two-wash condition is better than the no wash condition for RNA assays. When the same assays are performed with magnetic particles added after lysis at least three washes are needed to clean the nucleic acids and remove PCR inhibitors and later Cps are observed (data not shown), as compared to one or two washes performed with the magnetic particles in lysis. Without being bound to one theory, it is believed that faster sample preparation protocols that include magnetic particles during mechanical lysis may result in fewer inhibitors being captured by the magnetic particles, so fewer washes may be required compared to slower and longer binding processes when the magnetic particles are added exclusively after lysis is complete.

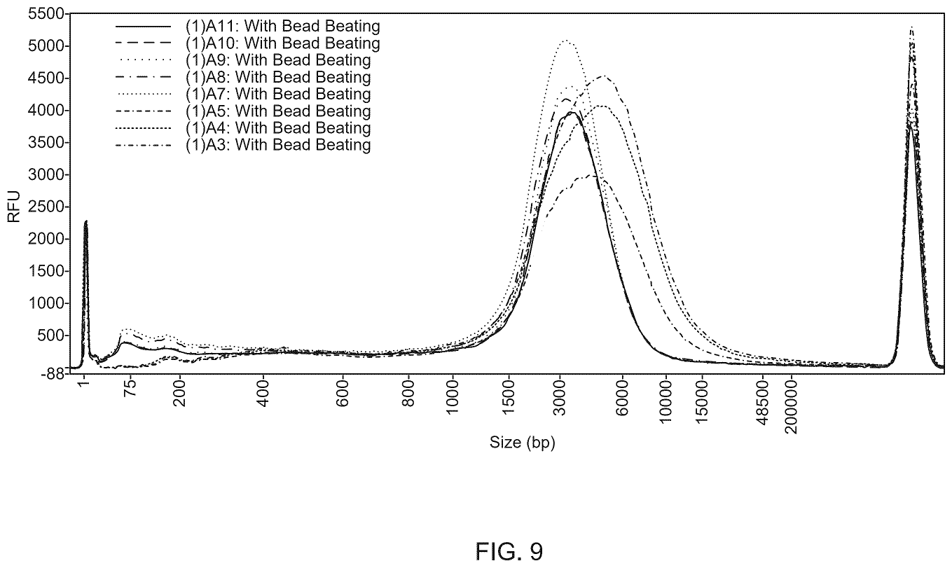

Typically, magnetic silica particles for nucleic acid recovery are added only after bead beating for lysis is complete. It has long been thought that having silica-coated magnetic silica particles present during manual lysis could damage the magnetic particles by breaking off the silica coating during lysis. If this damage were to happen, the nucleic acids would then bind to the free silica that is no longer associated with the magnetic core, and subsequent isolation of the magnetic particles would fail to recover the nucleic acids bound free silica. The inventors in this case have observed the opposite phenomenon. Recovery of nucleic acids appears to be more efficient when magnetic silica particles are included in lysis formation and the recovered nucleic acids are co-purified with fewer contaminants and they are more concentrated, as compared to nucleic acids obtained from lysed organisms by conventional methods. It has also been thought that bead beating magnetic particles with nucleic acids bound to them could excessively shear and damage the nucleic acids. If this damage were to happen, the nucleic acids could be compromised, and a smaller quantity would be available for amplification. Thus, the methods described herein are atypical and contrary to normal teachings in the art. Data presented herein (see, e.g., FIGS. 6A-8) demonstrate that adding the magnetic silica particles to the lysis can boost the recovery for nucleic acids from the sample and the nucleic acids are likely co-isolated with fewer PCR inhibitors. Data presented in FIG. 9 demonstrates that bead beating lysis in the presence of magnetic silica particles does not cause nucleic acid shearing. This means, that for a given assay, the unknown nucleic acids may be detected in a shorter period of time (e.g., after fewer cycles of amplification) or the limit of detection for a given cell type may be lower (i e., the assay for a given cell may be more sensitive because recovery of nucleic acids from the cells is boosted) or both.

Without being bound to theory, it is believed that improved recovery in samples where bead beating is done in the presence of some or all of the magnetic particles improves nucleic acid recovery due to better mixing and binding kinetics between the magnetic silica particles, the recovery of fewer proteins and other PCR inhibitors, the recovery of more nucleic acid, and the breaking up magnetic particle aggregates, which provides the nucleic acids with additional surface area for binding. It is also believed that the mechanical action of bead beating may alter the silica surface and increase surface area of the individual magnetic silica particles (e.g., through formation of microscopic scratches in the magnetic particle surface), thus increasing the likelihood of positive binding interactions between the magnetic silica particles and the nucleic acids. Also, because the contact time between the lysate and the magnetic particles is shorter when the magnetic particles are included in the lysis, the magnetic particles appear to co-isolate fewer PCR inhibitors with the nucleic acids. Again, without being bound to one theory, it is believed that the fast and rigorous motion during bead beating may change how nucleic acids or proteins and inhibitors bind to the magnetic particles, which may result in capturing more nucleic acids and fewer proteins and/or inhibitors when compared to protocols where the magnetic particles are added exclusively after lysis is complete.

Also, the protocols where some or all of the magnetic particles are present during mechanical lysis have certain speed advantages over a traditional protocol where magnetic particles are added subsequent to bead beating. These advantages include, but are not limited to, the breaking up aggregates, which means that fewer aggregates are likely to settle to the bottom of the container, making for faster and easier separation from the lysis particles. Likewise, the breaking up of aggregates likely increases the available surface area on the beads for nucleic acid binding. Additionally, because some or all of the magnetic particles are present during lysis, these protocols can reduce or eliminate time spent in rehydration or resuspension of the magnetic particles. Further, because the magnetic particles are present during lysis and are constantly being mixed, with increased interactions with the nucleic acids, nucleic acid binding can be much faster. Lysis in the presence of magnetic particles may also increase the likelihood that a given magnetic particle has nucleic acids bound thereto. On the other hand, because the binding kinetics are faster and the interaction time is correspondingly shorter when the magnetic particles are included in the lysis, the magnetic particles may be less likely to collect proteins and other inhibitors of downstream nucleic acid amplification. Furthermore, the lysate solution can heat up simply from friction during mechanical bead beating, and this heating can assist with faster diffusion kinetics and more efficient lysis, but this can be potentially deleterious for some binding kinetics, causing loosely bound particles to detach. Because lysis in the presence of magnetic particle can lead to shorter bead beating times and faster recovery, the sample is less likely to heat excessively and affect recovery.

The methods described herein suitably may including adding a first quantity of magnetic particles prior to mechanical lysis (e.g., bead beating) and then adding a second quantity of magnetic particles after mechanical lysis. It is believed that some fragments, illustratively double-stranded DNA, can be tightly bound to the magnetic particles or other silica surfaces and this binding can persist even throughout the lysis agitation steps. It is thought that these fragments would preferentially bind to the magnetic particles during the lysis and mixing phase. Other nucleic acids, such as single-stranded RNA, tend to bind more loosely, and binding may not survive the agitation steps. Adding a second quantity of magnetic particles with fresh nucleic acid binding sites can quickly and efficiently bind unbound nucleic acids post mechanical agitation. It is also thought that magnetic particles are not perfectly homogeneous and may have localized physical or chemical characteristics that favor binding nucleic acids in different configurations. Once a particular type of binding site is saturated, the bead may have a lost propensity for binding nucleic acids to the remaining binding sites. A fresh quantity of magnetic particles also provides a fresh injection of a greater variety of binding sites for any of the unbound nucleic acids to bind to.

Because the lysate solution can heat up simply from friction during mechanical bead beating and this can be deleterious, the fresh injection of cooler magnetic particles and subsequent airflow through the instrument to cool the solution can aid in stronger binding kinetics of loosely bound nucleic acid molecules. Thus, the combination of magnetic particle addition both before and after mechanical lysis advantageously promotes binding of those molecules that will tightly bind to the surfaces as well as those that are more loosely bound.

The foregoing methods described herein above suitably may further include capturing the magnetic particles with a magnet and using the magnet to transfer the magnetic particles to another container. Alternatively, the lysate containing the suspended magnetic particles may be flowed into another container where the magnetic particles can be extracted from the fluid. The spent lysate without the magnetic particles may then be flowed back into the lysis container or into another waste container. The methods described herein suitably may further include washing the nucleic acid binding magnetic particles with a wash buffer to remove waste material from the lysate from the magnetic particles. Preferably, the wash buffer can remove residual lysate, contaminants, and the like from the magnetic particles without eluting an appreciable amount of the nucleic acids from the magnetic particles. An example wash may include adding the wash buffer to the magnetic particles, gently agitating the magnetic particles for a period of time (e.g., a few seconds), recapturing the magnetic particles with a magnet, and removing the spent wash buffer from the container or transferring the magnetic particles to another container. For some sample types, no washing may be necessary. As such, the methods described herein may include zero washes. Typically, however, the methods described herein may include 1-3 washes. The washing performed in the methods described herein suitably may not include one or more of heating the wash buffer and/or magnetic particles prior to or during the washing, aggressively mixing the magnetic particles and the wash buffer, or incubating the magnetic particles and the wash buffer for a period of time greater than 1 second, 2 seconds, 3 seconds, 4 seconds, 5 seconds, 6 seconds, 7 seconds, 8 seconds, 9 seconds, 10 seconds, or any time therebetween. Preferably, a wash cycle may include incubating the magnetic particles and the wash buffer for a period of time of about 2-3 seconds.

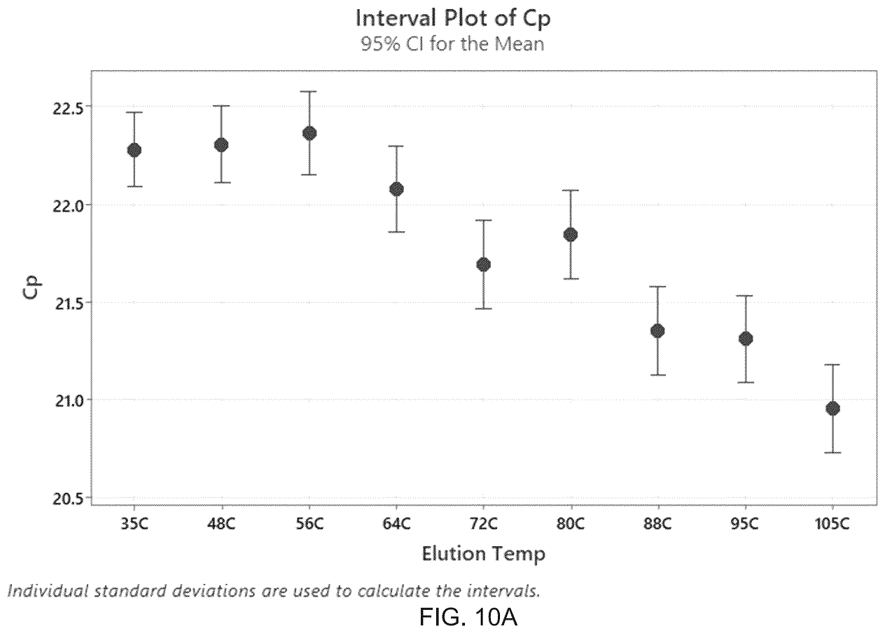

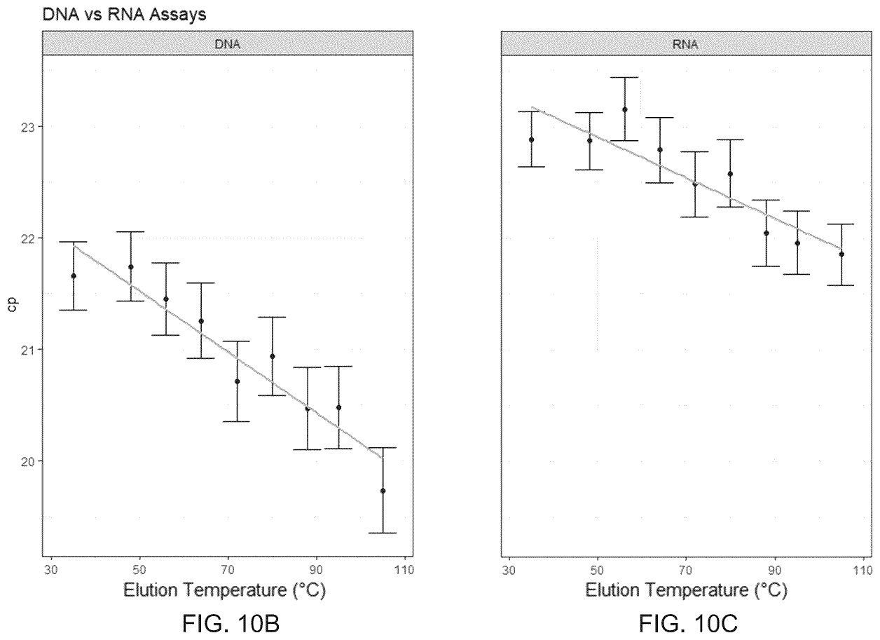

Following the wash, the methods described herein suitably may include releasing the magnetic particles from the capture magnet (either in the container after the wash buffer has been removed or in a new container) and adding an elution buffer to the magnetic particles, mixing the magnetic particles with the elution buffer, recapturing the magnetic particles with the magnet, and isolating the elution buffer from the magnetic particles. Optionally, the elution buffer may be heated prior to adding the elution buffer to the magnetic particles. This preheating can take place during the lysis and/or washing steps, so that the elution buffer is already heated at the time of elution. Illustratively, the elution buffer can be heated to a temperature between 35° C. and 105° C. (e.g., 50° C. to 100° C.) prior to mixing the elution buffer with the magnetic particles, depending on sample types and other factors. The inventors in the present case have found that heated elution temperature and duration can rapidly and efficiently elute RNA/DNA and leave proteins and inhibitors on the magnetic particles. This is illustrated, for example, in FIG. 10A which shows average Cp improvements for DNA and RNA assays as the pre-heated temperature of elution buffer is increased from about 35° C. to about 105° C. As can be seen in FIG. 10A, as the temperature of the elution buffer is increased from about 60° C. to about 100° C., the average Cp improved by up to about 1.5 to 2.5 Cp units. This represents about a 3-5 fold increase in the amount of nucleic acid eluted from the magnetic particles. FIGS. 10B and 10C show that the same trend exists for both DNA and RNA assays. The lines of best fit for the DNA and RNA data show that the average Cp improved by about 2 Cp units for DNA assays and about 1.5 Cp units for RNA assays. This represents about a 4-fold increase in the amount of DNA and about a 3-fold increase in the amount of RNA eluted from the magnetic particles.

While reference is made to moving the magnetic particles to a second chamber in various embodiments discussed herein, it is understood that an alternative embodiment is to retain the magnetic particles in the first chamber and remove the unbound lysate from this chamber.

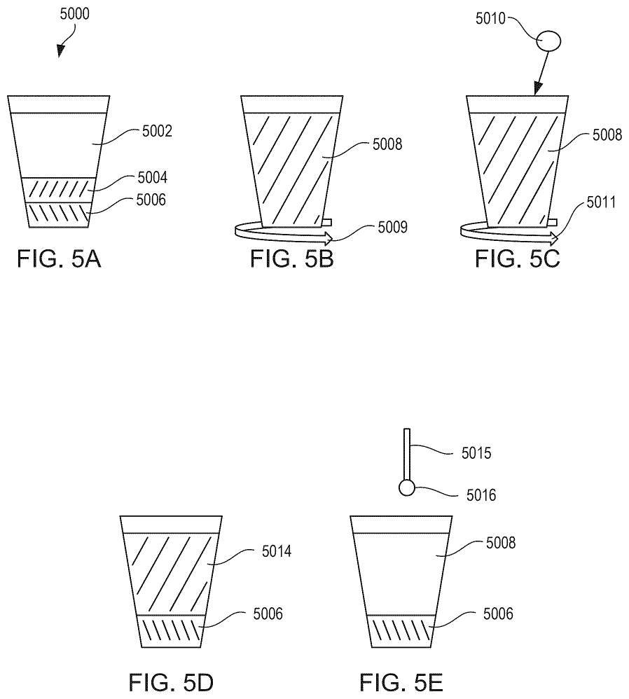

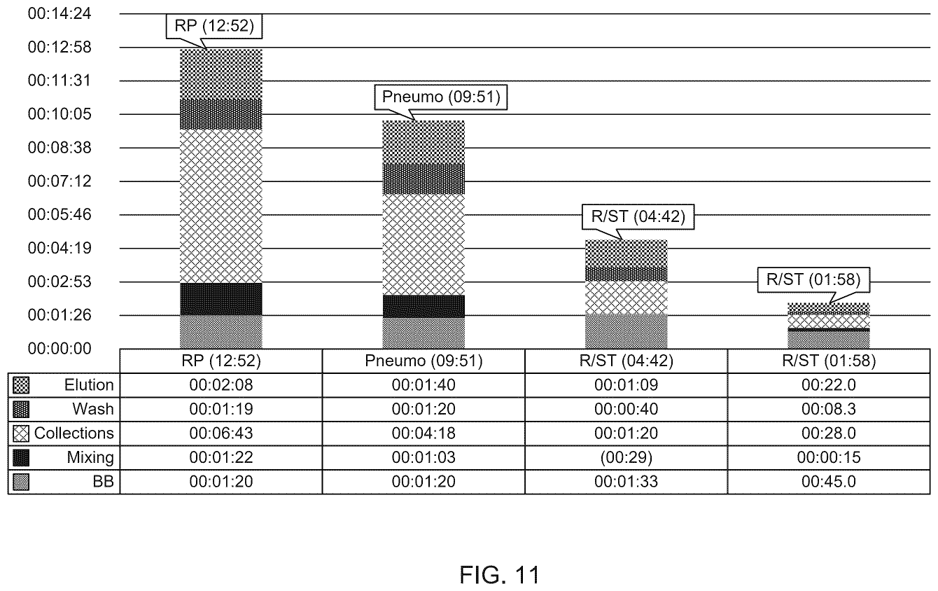

Referring now to FIGS. 5A-5E, an exemplary method of cell lysis and nucleic acid recovery is illustrated. As shown in FIG. 5A, a sample plus lysis buffer 5002 are combined in a container 5000 with lysis particles 5006 and magnetic particles 5004. As shown in FIG. 5B, the contents of the container 5000 suitably may be bead beaten (as depicted schematically by arrow 5009) for a sufficient time to create a lysate 5008. In one aspect, the bead beating may be made more efficient by compressing the container against the bead beating device with an appropriate amount of pressure. The time required to prepare a lysate can vary depending on factors such as, but not limited to, the sample material to be lysed, the quantity of sample, the speed or frequency of the bead beating motor, the pressure applied to the bead beater, and the bead lysis instrument. Reducing air from the chamber (illustratively via vacuum drawn on the container or by using the pressure applied to the container to expel air from the container) and bead beating under pressure helps to impart more energy to the sample and mitigate foam generation for more efficient lysis. Illustratively, a pressure regulated feedback control mechanism and electronically controlled motor may be used to deliver more power and adjust the lysis energy of the system to achieve shorter bead lysis times. Times of a few seconds to a few minutes are typical. With the bead beating instrument illustrated in FIG. 4 and the instrument and pouch of FIGS. 1 and 2, bead beating times of a few seconds may be sufficient (e.g., 1 second, 5 seconds, 10 seconds, 20 seconds, 25 seconds, 30 seconds, 40 seconds, 45 seconds, 50 seconds, 55 seconds to 1 minute). It is preferrable to bead beat with as much force and/or intensity as the system will allow and for no more than the time necessary to lyse the material in the sample. Not only does this save time in sample preparation, but too much bead beating time can shear nucleic acids and, eventually, degrade sample quality. This is illustrated in FIG. 11, which shows fragment sizes of detected human genomic DNA with or without bead beating for 120 seconds. With extended bead beating times, the largest fragments may be reduced in length and a higher concentration of smaller fragments may be detected.

The protocols discussed herein do not significantly shear nucleic acids or degrade sample quality. In general, harder to lyse organisms such as Cryptosporidium and yeasts prefer high RPM and long bead beating durations, whereas other organism such as E. coli and viral targets prefer lower RPMs combined with shorter bead beating durations. The optimal combinations of RPM and duration is assay specific. Optimizing the RPM and duration for easier to lyse organisms can leave difficult to lyse organisms unlysed; and optimizing for the most difficult to lyse organisms can result in shearing of the nucleic acids from the easy to lyse organisms. In addition to shearing, extended bead beating at elevated speeds can lead to heating of the sample, which can reduce yield. In general, it is best to optimize to a point that balances the lysis needs of all the organisms in a panel. Bead beating at a high speed (e.g., 10,000-12,000 RPM) for the shortest duration possible (e.g., 20-60 seconds) can maximize lysis efficiency and sample quality while saving considerable sample preparation time and overall time-to-result.

Returning to FIG. 5C, after bead beating, a second quantity of magnetic particles 5010 may be combined with the lysate 5008. The second quantity may be substantially the same as the first quantity, or it may be substantially larger or smaller. Illustratively, the amount of the first and second quantities of magnetic particles is substantially the same. Moreover, the first quantity and second quantity may be the same type of silica coated magnetic particles, or they may vary in the thickness of silica, as desired to bias the recovery of RNA, DNA, or both.

The second quantity of magnetic particles 5010 may, for example, be combined with the lysate 5008 by agitation (as schematically depicted by arrow 5011). Agitation 5011 may be effected by the bead beater used in the lysis step, although the duration of agitation may be shorter and less intense than the agitation used to produce the lysate. The first and second quantities of magnetic particles 5004 and 5010 can be incubated in the lysate 5008 for a selected period of time sufficient to capture the nucleic acids from solution (e.g., a few seconds to a few minutes). As illustrated in FIGS. 5D and 5E, the lysis particles 5006, which are typically much larger than the magnetic particles 5004 and 5010, may settle much faster than the magnetic particles. A magnet 5015 can be used to recover the magnetic particles 5004 and 5010 from the lysate 5008, as illustrated in 5E. In one embodiment, the magnetic particles 5004 and 5010 suitably may be kept in suspension to facilitate recovery by gently mixing the container. Since the lysis particles are much larger than that the magnetic particles, such gentle mixing may facilitate settling of the lysis particles while preferably keeping the magnetic particles in suspension and facilitating the separation of the magnetic particles from the lysis particles (not shown).

### Example 1

It has been found by the inventors in the present case that lysis and nucleic acid recovery are more efficient when lysis is performed in the presence of magnetic silica particles. This is illustrated, for example, in FIGS. 6A-7C. FIGS. 6A and 6B are bar graphs illustrating precent detection of organisms in sets of assays based on amplification from DNA (FIG. 7A) and sets of assays based on amplification from RNA (FIG. 7B) at various concentrations with (BB w/MB) or without (BB w/o MB) silica-coated magnetic particles present during bead beating lysis. FIG. 7A shows that percent detection for DNA-based assays is consistently better at 1×LOD, 0.1×LOD, and 0.01×LOD when bead beating is performed in the presence of silica-coated magnetic particles. RNA-based assays, however, appear to be about equivalent when silica-coated magnetic particles are added after lysis or when bead beating is performed in the presence of silica-coated magnetic particles.

Data in FIG. 7A show that the average Cp for the DNA and RNA assays where a first quantity of magnetic particles were added to the sample prior to forming the lysate and a second quantity of magnetic particles were added after lysis (w/Split Magbeads) was better than the control where the magnetic particles were added after forming the lysate. The improvement in Cp demonstrates that protocols ‘w/Split Magbeads’ improved by about 0.42 cycles of Cp, which represents an average of about a 1.34-fold increase in nucleic acid concentration when the magnetic particles were included in the lysis, as compared to controls where magnetic particles were added after lysis was completed. FIGS. 7B and 7C illustrate average Cp data for DNA-dependent assays (FIG. 7B) and RNA-dependent assays (FIG. 7C). FIG. 7B illustrates that DNA-dependent assays showed about a 1 Cp improvement, which represents an overall increase in DNA recovery from a lysate of about 2-fold when a first quantity of magnetic particles were added prior to formation of the lysate and a second quantity of magnetic particles were added after lysis. FIG. 7C illustrates that Cp performance for RNA recovered from magnetic particles present during lysis was about equivalent to the control. These data demonstrate that nucleic acids (i.e., DNA and RNA) recovered when lysis is performed in the presence of magnetic particles are more concentrated and likely of higher quality as compared to nucleic acids recovered when silica-coated magnetic particles are added after lysis.

This is surprising and unexpected because including magnetic silica particles in a mechanical lysis has traditionally been disfavored by persons of ordinary skill in the art because it has long been believed that having silica-coated magnetic particles present during mechanical lysis could damage the magnetic particles by breaking the silica coating off the particles. It was believed that loss of silica from the silica-coated magnetic particles could reduce the nucleic acid binding capacity of the magnetic particles and, in addition, the nucleic acids could be lost by binding to the unrecoverable bits of silica broken off the magnetic particles. Nevertheless, when organisms are mechanically lysed (e.g., by bead beating) in the presence of magnetic silica particles, nucleic acid binding to the magnetic silica particles can occur during lysis and nucleic acid binding may be more efficient and recovery may be greater. In fact, at least for DNA, the nucleic acid binding and recovery of nucleic acids appears to be significantly better. It also appears that the quality of the recovered nucleic acid is better when the magnetic silica particles are included in the lysis.

### Example 2

FIG. 8 compares the Cps for amplification of DNA ( [symbol] ) and RNA ( [symbol] ) recovered from magnetic particles included in the lysis with no washes, one wash, and two washes; except for the number of washes, the samples were identical. The no wash condition was clearly detrimental for both DNA and RNA assays, as compared to the one wash condition. This is likely due to the presence of PCR inhibitors co-isolated with the nucleic acids in the no wash condition. The one wash condition had improved Cps for both RNA and DNA assays, suggesting that one wash is sufficient to remove most PCR inhibitors without reducing the amount of nucleic acids that can be recovered from the magnetic particles. The two-wash condition appears that it may be slightly better for DNA assays and slightly worse for RNA assays, suggesting that some RNA may have been washed away. In any case, the two-wash condition is better than the no wash condition for RNA assays. When the same assays are performed with magnetic particles added after lysis at least three washes are needed to clean the nucleic acids and remove PCR inhibitors and later Cps are observed, as compared to one or two washes performed with the magnetic particles in lysis (data not shown).

### Example 3

Referring now to FIG. 11, bar charts are shown illustrating time savings for a number of specific examples in FilmArray sample preparation. In several years, the sample preparation time was reduced from ˜13 minutes to ˜2 minutes. That is an 11 minute reduction in sample preparation time. FilmArray is a state-of-the-art system and to be able to reduce the sample preparation time from ˜13 minutes to ˜2 minutes is surprising and unexpected. Comparing the RP2.1 time (4:42) to the R/ST time (1:58), time was again saved at every step. The R/ST time represents the time savings achievable using the methods described in the present application Comparing the RP2.1 sample preparation time to the R/ST sample preparation time shows an absolute time difference of 2:44 and a 58% time reduction. As can be seen in FIG. 11, time was saved in every sample preparation step, particularly in collections the magnetic particles from the lysate and in reductions to bead beating time. Nucleic acid release from cells may be accelerated by combining multiple lysis mechanisms with pressure control and motor feedback mechanisms. The tighter control and reduced variation also allow for decreased buffering times to ensure robust performance with the easiest to most difficult to lyse organism types. Binding and magnetic bead recovery were the most dramatically reduced from 7 minutes to 30 seconds by performing multiple steps in parallel and increasing the kinetics and efficiency of nucleic acids binding to magnetic particles and magnetic particles binding to the magnet. Comparing only the bead beating and magnetic particle collection times between RP2.1 and R/ST shows a reduction from 2:53 in RP2.1 to 1:13 in R/ST, absolute time savings of 1:40, and a 57% time reduction for just these two steps. Bead beating in the presence of magnetic particles allows for faster binding of nucleic acids and faster recovery of the nucleic acids from the lysate. Wash time was reduced from 40 seconds to approximately 8 seconds and elution time was reduced from 1:09 to 22 seconds.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. While certain embodiments and details have been included herein and in the attached invention disclosure for purposes of illustrating the invention, it will be apparent to those skilled in the art that various changes in the methods and apparatus disclosed herein may be made without departing from the scope of the invention, which is defined in the appended claims. All changes which come within the meaning and range of equivalency of the claims are to be embraced within their scope.

What is claimed is:

1. A method for cell lysis and nucleic acid recovery, comprising:
providing a sample container, a quantity of lysis particles, a sample suspected of containing one or more target nucleic acids, a lysis buffer, and a first quantity of nucleic acid binding magnetic particles,
disposing in the container the sample, the quantity of lysis particles, the lysis buffer, and the first quantity of nucleic acid binding magnetic particles,
agitating the container with the lysis particles, the sample, the lysis buffer, and the first quantity of nucleic acid binding magnetic particles for a first period of time to generate a lysate,
dispersing a second quantity of nucleic acid binding magnetic particles into the lysate in the container, and
recovering the first and second quantities of nucleic acid binding magnetic particles from the lysate,
wherein adding the first quantity of nucleic acid binding magnetic particles and then the second quantity of nucleic acid binding magnetic particles is selected to boost recovery of nucleic acids from the sample.

2. The method of claim 1, wherein the lysis buffer comprises a buffering agent, a chaotropic salt, and a non-ionic surfactant.

3. The method of claim 2, wherein the lysis buffer is an aqueous buffer comprising the buffering agent, 50-60% of the chaotropic agent, and 10-20% of the non-ionic surfactant.

4. The method of claim 3, wherein the chaotropic agent is a guanidinium salt and the non-ionic surfactant is one of Triton X-100, polidocanol (Thesit), Triton X-114, NP-40, Arlasolve 200, Brij 010, octyl β-D-glucopyranoside, a saponin, nonaethylene glycol monododecyl ether, and combinations thereof.

5. The method of claim 1, wherein the recovering further comprises one of releasing the first and second quantities of nucleic acid binding magnetic particles recovered from the lysate into another sample container, or removing the lysate and lysis particles from the container and releasing the first and second quantities of nucleic acid binding magnetic particles recovered from the lysate back into the container.

6. The method of claim 5, further comprising washing the first and second quantities of nucleic acid binding magnetic particles with a wash buffer, recapturing the first and second quantities of nucleic acid binding magnetic particles with the magnet, and removing the wash buffer.

7. The method of claim 6, wherein the washing does not include one or more of heating the wash buffer and magnetic particles prior to or during the washing, aggressively mixing the magnetic particles and the wash buffer, or incubating the magnetic particles and the wash buffer for a period of time greater than 10 seconds.

8. The method of claim 6, further comprising releasing the first and second quantities of nucleic acid binding magnetic particles from the magnet, adding an elution buffer to the first and second quantities of nucleic acid binding magnetic particles, releasing the magnet, and mixing the magnetic particles with the elution buffer, recapturing the first and second quantities of nucleic acid binding magnetic particles with the magnet, and transferring the elution buffer to another sample container.

9. The method of claim 6, further comprising adding an elution buffer to the first and second quantities of nucleic acid binding magnetic particles and mixing the magnetic particles with the elution buffer, recapturing the first and second quantities of nucleic acid binding magnetic particles with the magnet, and transferring the first and second quantities of nucleic acid binding magnetic particles to another sample container.

10. The method of claim 8, wherein the elution buffer is configured to elute nucleic acids captured by the nucleic acid binding magnetic particles into the elution buffer.

11. The method of claim 1, wherein an amount of the second quantity of nucleic acid binding magnetic particles is substantially equal to an amount of the first quantity of nucleic acid binding magnetic particles.

12. The method of claim 8, further comprising assaying the elution buffer for presence of the one or more target nucleic acids suspected to be in the sample.

13. The method of claim 12, wherein the assaying comprises a nucleic acid amplification step and a step of detection of amplified nucleic acids produced in the nucleic acid amplification step.

14. The method of claim 1, wherein the agitating step comprises heating the sample and the dispersing step comprises cooling the sample.

15. A method for cell lysis and nucleic acid recovery, comprising:
providing a sample container,
combining in the sample container a quantity of lysis particles, a first quantity nucleic acid binding magnetic particles, a sample suspected of containing one or more target nucleic acids, and a lysis buffer,
agitating the lysis particles, the sample, the lysis buffer, and the first quantity of nucleic acid binding magnetic particles in the sample container for a period of time sufficient to generate a lysate,
dispersing a second quantity of nucleic acid binding magnetic particles into the lysate in the sample container,
incubating the first and second quantities of nucleic acid binding magnetic particles in the lysate for a period of time, and
capturing the first and second quantities of nucleic acid binding magnetic particles from the lysate using a magnet,
wherein adding the first quantity of nucleic acid binding magnetic particles and then the second quantity of nucleic acid binding magnetic particles is selected to boost recovery of nucleic acids from the sample.

16. The method of claim 15, wherein the period of time of incubating the first and second quantities of nucleic acid binding magnetic particles in the lysate is in a range of 1 second to 1 minute, preferably in a range of 20-30 seconds.

17. (canceled)

18. The method of claim 15, wherein capturing the first and second quantities of magnetic particles from the lysate further comprises transferring the magnetic particles to a second sample container and releasing the magnetic particles from the magnet into the second sample container, or removing the lysate and the lysis particles from the first sample container and releasing the magnetic particles from the magnet back into the first sample container.

19-25. (canceled)

26. A method for cell lysis and nucleic acid recovery, comprising:
providing a sample container comprising a plurality of fluidly connected reaction chambers including a sample lysis chamber, a nucleic acid recovery chamber, and at least a first nucleic acid amplification chamber,
combining in the sample lysis chamber a quantity of lysis particles, a first quantity nucleic acid binding magnetic particles, a sample suspected of containing one or more target nucleic acids, and a lysis buffer,
agitating the lysis particles, the sample, the lysis buffer, and the first quantity of nucleic acid binding magnetic particles in the lysis chamber for a period of time sufficient to generate a lysate,
mixing a second quantity of nucleic acid binding magnetic particles into the lysate, transferring at least a portion of the lysate having the first and second quantities of nucleic acid binding magnetic particles suspended therein to the nucleic acid recovery chamber,
using a magnet, capturing the first and second quantities of nucleic acid binding magnetic particles from the lysate in the nucleic acid recovery chamber,
removing the lysate but not the first and second quantities of nucleic acid binding magnetic particles from the nucleic acid recovery chamber,
releasing the first and second quantities of nucleic acid binding magnetic particles from the magnet and washing the first and second quantities of nucleic acid binding magnetic particles with a wash buffer in the nucleic acid recovery chamber,
recapturing the first and second quantities of nucleic acid binding magnetic particles with the magnet and removing the wash buffer,
releasing the magnet and mixing the first and second quantities of nucleic acid binding magnetic particles with an elution buffer in the nucleic acid recovery chamber to elute the nucleic acids from the magnetic particles,
recapturing the first and second quantities of nucleic acid binding magnetic particles with the magnet and transferring the elution buffer to the first nucleic acid amplification chamber,
wherein adding the first quantity of nucleic acid binding magnetic particles and then the second quantity of nucleic acid binding magnetic particles is selected to boost recovery of nucleic acids from the sample.

27. The method of claim 26, wherein capturing the nucleic acid binding magnetic particles comprises deploying a magnet adjacent to the nucleic acid recovery chamber to contain the magnetic particles in that chamber.

28. The method of claim 26, further comprising combining the elution buffer with reagents for a nucleic acid amplification reaction to form an amplification mix in the first nucleic acid amplification chamber and subjecting the amplification mix to amplification conditions to assay for presence of the one or more target nucleic acids suspected to be in the sample.

29-38. (canceled)
